(19) United States
(12) Patent Application Publication
(10) Pub. No.: US 2019/0211099 A1
(43) Pub. Date: Jul. 11, 2019

(54) METHODS AND COMPOSITIONS FOR TREATING CANCER WITH SIGLEC-9 ACTIVITY MODULATORS

(71) Applicant: KING'S COLLEGE LONDON, Strand, London (GB)

(72) Inventors: Joy Marilyn BURCHELL, London (GB); Richard Esmond BEATSON, London (GB)

(21) Appl. No.: 16/312,716

(22) PCT Filed: Jun. 30, 2017
(86) PCT No.: PCT/GB2017/051917

§ 371 Date: Dec. 21, 2018

(30) Foreign Application Priority Data
Jul. 1, 2016 (GB) 1611535.4

(51) Int. Cl. C07K 16/28; A61K 39/395; G01N 33/574; A61K 45/06; A61P 35/00

(52) U.S. Cl. CPC C07K 16/2803; A61K 39/39558; G01N 33/57492; G01N 2333/4725; A61P 35/00; C07K 2317/76; A61K 45/06

(57) ABSTRACT

The invention provides methods and compositions for treating Siglec-9 mediated cancer in a subject, where the cells of the cancer express sialylated Core-1-MUC1 glycoproteins that engage with Siglec-9 expressed on certain immune cells, for example, monocytes and macrophages of the subject. Prior to treatment, the cancerous cells may evade the immune system of the host by binding Siglec-9 expressed by the immune cells, whereupon binding activates a number of pro-tumorigenic, Siglec-9 mediated activities in or via the immune cells. However, when treated with an inhibitor of Siglec-9 activity, the Siglec-mediated activities can be mitigated and the host immune system can recognize and elicit an immune response against the cancer cells expressing the sialylated Core-1 MUC1 glycoproteins.

### CROSS REFERENCE TO RELATED APPLICATIONS

This application claims the benefit of, and priority to, Great Britain Patent Application No. 1611535.4, filed Jul. 1, 2016, the entire contents of which are hereby incorporated by reference in their entirety.

### FIELD OF THE INVENTION

The invention relates generally to methods and compositions for treating cancer in a subject, and, more particularly, the invention relates to methods and compositions for treating Siglec-9 mediated cancer in the subject.

### BACKGROUND

Over the years it has been observed that cancers have developed a variety of mechanisms for evading an immune response elicited against a cancer in a subject. In certain cases, the cancer cells can initiate a pro-tumorigenic, permissive local environment. For cancer cells to remodel their microenvironment, they often need to elicit changes in a subject that include the recruitment and education of monocytes, and the repolarization of resident macrophages (Quail et al. (2013) NAT. MED. 19: 1423-1437). Macrophages are phenotypically plastic and factors produced by cancer cells often can polarize macrophages to become tumor-promoting. These tumor-educated macrophages promote the growth and invasion of cancer cells by contributing to all the stages involved in cancer dissemination, cumulating in metastasis (Kitamura et al. (2015) NAT. REV. IMMUNOL. 15: 73-86) and poor prognosis (Gentles et al. (2015) NATURE MEDICINE 21(8):938-45).

Changes in glycosylation occur in essentially all types of cancers and changes in mucin-type O-linked glycans are the most prevalent aberrant glycophenotype when increased sialylation often occurs (Pinho et al. (2015) NAT. REV. CANCER 15: 540-555; Burchell et al. (2001) J. MAMMARY GLAND BIOL. NEOPLASIA 6: 355-364). The transmembrane mucin MUC1 is upregulated in breast and the majority of adenocarcinomas and, due to the presence of a variable number of tandem repeats that contain the O-linked glycosylation sites, can carry from 100 to over 750 O-glycans (Gendler et al. (1990) J. BIOL. CHEM. 265: 15286-93). The aberrant glycosylation seen in cancer results in the multiple O-linked glycans carried by MUC1 being mainly short and sialylated (Pinho et al. (2015) supra; Burchell et al. (1999) GLYCOBIOLOGY 9: 1307-11) in contrast to the long, branched chains seen on MUC1 expressed by normal epithelial cells (Lloyd et al. (1996) J. BIOL. CHEM. 271: 33325-34). In carcinomas, the aberrant O-linked glycosylation of MUC1 can alter the interaction of MUC1 with lectins of the immune system (Beatson et al. (2015) PLoS ONE 10: e0125994) and thereby influence tumor-immune interplay.

Siglecs (sialic acid-binding immunoglobulin-like lectins) are a family of sialic acid binding lectins, which, with the exception of Siglec-4, are expressed on various cells of the immune system (Macauley et al. (2014) NAT. REV. IMMUNOL. 14: 653-666). The cytoplasmic domains of most Siglecs contain immunoreceptor tyrosine-based inhibitory motifs (ITIMs), which recruit the tyrosine phosphatases, SHP1 and/or SHP2 (Avril et al. (2004) J. IMMUNOL. 173:6841-6849) and so regulate the cells of the innate and adaptive immune response (Crocker (2007) NAT. REV. IMMUNOL. 7: 255-266). It has recently become apparent that certain Siglecs play a role in cancer immune suppression, the hypersialylation seen in cancers inducing binding to these lectins (Jandus et al. (2014) J. CLIN. INVEST. 124: 1810-1820; Laubli et al. (2014) PROC. NATL. ACAD. SCI. USA 111: 14211-14216; Hudak et al. (2014) NAT. CHEM. BIOL. 10: 69-75).

Despite the significant advances being made in cancer treatment and management, there is still an ongoing need for new and effective therapies for treating and managing cancer.

### SUMMARY OF THE INVENTION

The invention is based, in part, upon the discovery that cancer cells in a subject that express certain sialylated Core-1-MUC1 glycoproteins not expressed by normal epithelial cells can modulate the tumor immune microenvironment through the engagement of Siglec-9 expressed on the surface of certain myeloid cells, for example, monocytes and macrophages. Siglec-9 is a sialic acid binding lectin predominantly expressed on myeloid cells that are able to negatively regulate the immune responses. The cancer cells expressing such sialylated Core-1-MUC1 glycoproteins, can, through the engagement of Siglec-9, educate the myeloid cells to release factors that influence the tumor microenvironment and promote disease progression, and to induce tumor-associated macrophages (TAMs) to show increased expression levels of the immune checkpoint ligand PD-L1, indoleamine 2,3-dioxygenase (IDO), the scavenger receptor CD163 and the mannose receptor CD206. CD206 and CD163 are tumor-associated macrophage markers. Therefore, as used herein, the expression ‘tumor-associated macrophage’ or ‘TAM’ refer to macrophages which express the CD206 and/or CD163 markers, and/or increased expression of PD-L1 and/or IDO as compared to resting tissue resident or inflammatory macrophages or macrophages not exposed to MUC1-ST as illustrated hereinafter. Examples of resting tissue resident macrophages are M-CSF monocyte derived macrophages.

As a result, the cancer cells expressing such sialylated Core-1-MUC1 glycoproteins can not only evade the immune system of the host subject but can also induce the differentiation of monocytes and macrophages into anti-inflammatory, pro-tumorigenic TAMs. It has been discovered that these pro-tumorigenic effects can be mitigated or reversed by inhibiting Siglec-9 activity in the monocytes and macrophages. As a result, these discoveries can facilitate new and effective cancer therapies.

In one aspect, the invention provides a method of treating cancer in a subject, for example, a human subject, in need thereof. The method comprises administering to the subject an effective amount of an inhibitor of Siglec-9 activity thereby to treat the cancer in the subject where the cancer has been identified as comprising cancerous cells that express one or more sialylated Core-1-MUC1 glycoproteins. As a result the subject suitable for such treatment is characterized or identified as having a cancer comprising cancerous cells that express one or more sialylated Core-1-MUC1 glycoproteins, for example, MUC1-ST, MUC1-diST, or a combination thereof, either alone or in association with one or more other MUC1 glycoproteins comprising a glycan other than a Core-1 glycan, such as a Core-2 glycan. The glycoproteins may be secreted from the cancerous cells and/or expressed on the cell surface of the cancerous cells.

It is contemplated a variety of inhibitors of Siglec-9 activity may be used in the practice of this aspect of the invention. In certain embodiments, the inhibitor acts by blocking, reducing or otherwise neutralizing binding between sialylated Core-1-MUC1 glycoprotein (e.g., MUC1-ST and/or MUC1-DiST) and Siglec-9. For example, the inhibitor may be an antibody, for example, an anti-Siglec-9 antibody, a nucleic acid, for example, a Siglec-9 aptamer or spiegelmer, or an anti-sense molecule, or a small molecule, for example, a MEK/ERK inhibitor or a calcium flux inhibitor, or a combination thereof. In one embodiment, the inhibitor is an anti-Siglec-9 neutralizing antibody. Exemplary anti-Siglec-9 antibodies may have a binding affinity stronger than 1 nM for Siglec-9. The antibody may be a humanized or a human antibody, and may have a human IgG1, IgG2, IgG3, IgG4, or IgE isotype. In certain embodiments, the antibody has a human IgG4 isotype. The anti-Siglec-9 antibody may act to prevent the binding of the glycoprotein expressed by the cancerous cell (e.g., a sialylated Core-1-MUC1 glycoprotein) to Siglec-9 expressed by a monocyte, macrophage, or neutrophil.

It is contemplated that the method can be used to treat a variety of cancers including, for example, breast, colon, colorectal, lung, ovarian, pancreatic or prostate cancer, as well as cervical, endometrial, head and neck, liver, renal, skin, stomach, testicular, thyroid or urothelial cancer. Furthermore, the cancer may be an adenocarcinoma. Furthermore, the cancer may be a metastatic cancer and/or a refractory cancer.

Given that cancer cells expressing such sialylated Core-1-MUC1 glycoproteins, can, through the engagement of Siglec-9, induce the differentiation of myeloid cells into tumor-associated macrophages (TAMs) showing increased expression levels of the immune checkpoint ligand PD-L1 and IDO, the method may further comprise administering an IDO inhibitor, or an immune checkpoint inhibitor, for example, a PD-1 inhibitor, PD-L1 inhibitor, CTLA-4 inhibitor, adenosine A_{2A} receptor inhibitor, B7-H3 inhibitor, B7-H4 inhibitor, BTLA inhibitor, KIR inhibitor, LAG3 inhibitor, TIM-3 inhibitor, VISTA inhibitor or TIGIT inhibitor in combination with a Siglec-9 inhibitor.

Thus the invention further provides an inhibitor of Siglec-9 activity for use in the treatment of cancer, wherein the cancer comprises, or has been identified as comprising, cancerous cells that express one or more sialylated Core-1-MUC1 glycoproteins. In particular the inhibitor is for use in methods as described above. Where the inhibitor is used in combination with an IDO inhibitor or an immune checkpoint inhibitor as described above, these may be administered together (simultaneously or sequentially) depending upon usual clinical practice. Combinations of an inhibitor of Siglec-9 activity and an IDO inhibitor or an immune checkpoint inhibitor, which may be present in a single unitary formulation or in multiple formulations, for example in a kit, form yet a further aspect of the invention.

In another aspect, the invention provides a method of reducing PDL-1 or IDO expression in a monocyte, macrophage, or neutrophil that expresses Siglec-9 and is capable of binding a sialylated Core-1-MUC1 glycoprotein (for example, MUC1-ST, MUC1-diST, or a combination thereof, either alone or in association with other MUC1 glycoproteins comprising other different glycans such as Core-2 glycans), expressed by a cancerous cell, for example, a human cancerous cell. The method comprises contacting the monocyte, macrophage, or neutrophil with an inhibitor of Siglec-9 activity thereby to reduce PDL-1 or IDO expression in the monocyte, macrophage, or neutrophil. The glycoprotein may be secreted from the cancerous cell and/or expressed on the cell surface of the cancerous cell.

It is contemplated a variety of inhibitors of Siglec-9 activity may be used in the practice of this aspect of the invention. In certain embodiments, the inhibitor acts by blocking, reducing or otherwise neutralizing binding between sialylated Core-1-MUC1 glycoprotein (e.g., MUC1-ST and/or MUC1-DiST) and Siglec-9. For example, the inhibitor may be an antibody, for example, an anti-Siglec-9 antibody, a nucleic acid, for example, a Siglec-9 aptamer or spiegelmer, or an anti-sense molecule, or a small molecule, for example a MEK/ERK inhibitor or a calcium flux inhibitor, or a combination thereof.

In one embodiment, the inhibitor is an anti-Siglec-9 neutralizing antibody. Exemplary anti-Siglec-9 antibodies may have a binding affinity stronger than 1 nM for Siglec-9. The antibody may be a humanized antibody or a human antibody, and may have a human IgG1, IgG2, IgG3, IgG4, or IgE isotype. In certain embodiments, the antibody has a human IgG4 isotype or another isotype that elicits little or no antibody-dependent cell-mediated cytotoxicity (ADCC). The anti-Siglec-9 antibody may act to prevent the binding of the glycoprotein expressed by the cancerous cell (e.g., a sialylated Core-1-MUC1 glycoprotein) to Siglec-9 expressed by a monocyte, macrophage, or neutrophil.

In another embodiment, the inhibitor is a small molecule, for example a MEK/ERK inhibitor or a calcium flux inhibitor. Examples of such molecules are known in the art but include for example, trametinib, verapamil, diltiazem, nifedipine, nicardipine, isradipine, felodipine, amlodipine, nisoldipine, clevidipine, and nimodipine.

It is contemplated that the cancerous cells may be derived from a variety of cancers and cancerous tissues including, for example, breast, colon, colorectal, lung, ovarian, pancreatic, or prostate cancer, as well as cervical, endometrial, head and neck, liver, renal, skin, stomach, testicular, thyroid or urothelial cancer. The cancerous cell may be an adenocarcinoma. The cancerous cell may be derived from or associated with a metastatic cancer and/or derived from or associated with a refractory cancer.

Given that cancer cells expressing such sialylated Core-1-MUC1 glycoproteins, can, through the engagement of Siglec-9, induce the differentiation of myeloid cells into tumor-associated macrophages (TAMs) showing increased expression levels of PD-L1 and IDO, the method may further comprise contacting the monocyte or macrophage with an IDO inhibitor, or an immune checkpoint inhibitor, for example, a PD-1 inhibitor, PD-L1 inhibitor, CTLA-4 inhibitor, adenosine A_{2A} receptor inhibitor, B7-H3 inhibitor, B7-H4 inhibitor, BTLA inhibitor, KIR inhibitor, LAG3 inhibitor, TIM-3 inhibitor, VISTA inhibitor or a TIGIT inhibitor in combination with a Siglec-9 inhibitor.

In another aspect, the invention provides a method of identifying a subject with cancer likely to respond to treatment with an inhibitor of Siglec-9 activity. The method comprises determining whether the cancer comprises cancerous cells that express one or more sialylated Core-1-MUC1 glycoproteins (for example, MUC1-ST, MUC1-diST, or a combination thereof either alone or in association with other MUC1 glycoproteins comprising other different glycans such as Core-2 glycans). The glycoprotein may be secreted from the cancerous cell and/or expressed on the cell surface of the cancerous cell. It is contemplated that the cancerous cells may be derived from a variety of cancers and cancerous tissues including, for example, breast, colon, colorectal, lung, ovarian, pancreatic, or prostate cancer as well as cervical, endometrial, head and neck, liver, renal, skin, stomach, testicular, thyroid or urothelial cancer. The cancerous cell may be an adenocarcinoma, metastatic cancer, refractory cancer, or a combination thereof. It is contemplated that the subject may be a human subject. Such a method can be performed on cancerous cells initially present in a tissue or body fluid sample harvested from the subject. Once a subject has been identified as likely to respond to treatment with an inhibitor or Siglec-9 activity, the subject may be treated with one or more inhibitors of Siglec-activity, such as one or more of the inhibitors described herein, such as an anti-Siglec-9 antibody that prevents or otherwise reduces the binding of Siglec-9 and its cognate ligand, namely, the Core-1-MUC1 glycoprotein, so as to treat the cancer.

Determination of the expression of one or more sialylated Core-1-MUC1 glycoproteins by the cancerous cells can be carried out using techniques known in the art including antibody based techniques as described further hereinafter.

In each of the foregoing aspects, the Siglec-9 may be expressed by a monocyte, macrophage, or neutrophil in a subject. Furthermore, in each of the foregoing aspects, the inhibitor prevents differentiation of a macrophage into a tumor-associated macrophage (TAM). The inhibitor may induce the macrophage to differentiate into a pro-inflammatory macrophage and/or may prevent the loss of pro-inflammatory activity and/or may prevent the differentiation of a macrophage into a pro-tumorigenic macrophage. The inhibitor may reduce upregulation of PD-L1, IDO, CD163, and CD206 expression in myeloid cells educated by engagement with the sialylated Core-1-MUC1 glycoprotein. Understanding the mechanisms that contribute to immune suppression by myeloid cells will facilitate the development of new myeloid checkpoint inhibitors useful in immunotherapy, such as anti-Siglec 9 immunotherapy.

It is understood that in each of the foregoing methods of treating cancer described herein, the subject may be identified by any one of the methods of identifying a subject likely to respond to a treatment described herein.

These and other aspects and features of the invention are described in the following detailed description and claims.

### BRIEF DESCRIPTION OF THE DRAWINGS

The foregoing and other objects, features and advantages of the invention will become apparent from the following description of preferred embodiments, as illustrated in the accompanying drawings.

FIG. 1 is a schematic representation of aberrantly glycosylated forms of MUC1 expressed by cancerous cells.

FIGS. 2A-2J demonstrate that MUC1 carrying sialylated Core-1 glycans (MUC1-ST) bind to monocytes and macrophages through Siglec-9. FIG. 2A is a scatter plot showing binding of biotinylated MUC1-ST to isolated or differentiated immune cell subsets as determined by flow cytometry (n=4 independent donors). MFI was calculated against streptavidin-PE (SAPE) alone. FIG. 2B depicts fluorescence microscopy images showing U937 cells incubated with biotinylated MUC1-T or MUC1-ST, plus SAPE. FIGS. 2C and 2D are a scatter plots showing binding of biotinylated MUC1 glycoforms to donor monocytes (FIG. 2C) or monocyte derived macrophages (FIG. 2D) as determined by flow cytometry (n=11 independent donors). FIG. 2E is a bar graph showing binding of monocytes to MUC1 glycoforms in the presence of Ca′ or EDTA as determined by flow cytometry (n=3 independent donors). FIG. 2F is a bar graph showing binding of biotinylated MUC1 glycoforms to a panel of Siglec fusion proteins. FIG. 2G is a scatter plot showing inhibition of binding of MUC1-ST to monocytes by an anti-Siglec-9 antibody (n=7 independent donors). FIGS. 2H and 2I are a representative histograms showing MUC1-ST binding to monocytes (FIG. 2H) or monocyte-derived macrophages (FIG. 2I) after preincubation with an anti-Siglec-9 antibody (indicated by arrow) or isotype control (dark black solid line). The light black line is a control with SAPE alone. FIG. 2J depicts images of FFPE T47D cells (MUC1-ST+ve) stained with human Siglec-9 IgG fusion, anti-MUC1 antibody (HMFG2), or appropriate controls, and visualised using DAB. Scale bars represent 25 μm. Wherever indicated, * corresponds to p<0.05, ** to p<0.01, and *** to p<0.001 using paired or unpaired t-test where appropriate.

FIGS. 3A-3G demonstrate MUC1-ST binding to Siglec-9. FIGS. 3A and 3B are line graphs depicting a time course (FIG. 3A) and the concentration dependence (FIG. 3B) of MUC1-ST binding to CD14+ monocytes isolated from PBMCs as determined by flow cytometry (n=2 independent donors). FIG. 3C is a bar graph showing MUC1-ST binding to CD14+ monocytes isolated from PBMCs with or without neuraminidase treatment as determined by flow cytometry (n=3 independent donors). FIG. 3D is a bar graph showing staining of isolated monocytes (n=3 independent donors), M-CSF differentiated monocyte-derived macrophages (n=2 independent donors) and THP-1 cells with antibodies to the indicated Siglecs as determined by flow cytometry. Mean expression levels are shown. FIG. 3E is a line graph depicting the binding of MUC1-ST to isolated monocytes treated with indicated concentrations of antibodies to Siglecs 3, 7 and 9. The graph shows % binding inhibition for the indicated antibodies. FIG. 3F is a line graph depicting binding of isolated monocytes to biotinylated MUC1-ST or biotinylated polyacrylamide carrying the ST glycan in the presence of competing anti-Siglec-9 antibody at indicated concentrations. The graph shows % binding inhibition for the anti-Siglec-9 mAb (n=2 independent donors). FIG. 3G shows representative histograms for binding of MUC1-ST (dotted arrow) or PAA-ST to isolated monocytes or U937 cells in the presence of anti-Siglec-9 (solid arrow) or isotype antibodies.

FIGS. 4A-4J demonstrate that MUC1-ST binds to monocytic cell lines in a Siglec-9 dependent manner. FIGS. 4A and 4B are line graphs depicting a time course (FIG. 4A) and the concentration dependence (FIG. 4B) of MUC1-ST binding to THP-1 cells. FIGS. 4C-4G are line graphs depicting the binding of MUC1-ST to THP-1 cells (FIG. 4C), U937 cells (FIG. 4D), isolated monocytes (FIG. 4E), isolated neutrophils (FIG. 4F) and isolated macrophages (FIG. 4G) treated with the indicated concentrations of antibodies to Siglec-9. The graph shows % binding inhibition for the indicated antibody. FIG. 4H is a bar graph showing PAI-1 release from differentiated THP-1 cells in the presence of MUC1-ST/T as determined by ELISA. FIG. 4I is a bar graph showing concentrations of PAI-1, M-CSF and kynurenine in the supernatants of THP-1 cells treated with MUC1-ST in the presence of DMSO or PD98059. FIG. 4J is a line graph showing a time course of calcium flux in differentiated THP-1 cells treated with MUC1-ST/T as assayed by an intracellular fluorescent calcium reporter.

FIG. 5 is a table summarizing the percent inhibition of MUC1-ST binding to monocytes or macrophages by the indicated antibodies. N is shown in brackets. % inhibition was calculated by change in M.F.I. from control.

FIGS. 6A-6I demonstrate that MUC1-ST can induce monocytes to secrete factors associated with immune recruitment, microenvironment remodeling and tumor growth in a Siglec-9 dependent manner. FIG. 6A shows a protein array following treatment of isolated monocytes with MUC1-ST (bottom panel) or PBS control (top panel). Highlighted factors are as follows: 1—CXCL5; 2—Chitinase 3-like 1; 3—IL-8; 4—CCL3; 5—IL17A; 6—MMP-9; 7-CCL2; 8—PAI-1; 9—IL6; 10—CXCL1. FIGS. 6B-6D are bar graphs showing IL-6 release (FIG. 6B), M-CSF release (FIG. 6C), and PAI-1 release (FIG. 6D) by monocytes in response to MUC1-ST in a sialic acid dependent manner, as determined by ELISA.

FIGS. 6E-6G are bar graphs showing IL-6 release (FIG. 6E), M-CSF release (FIG. 6F), and PAI-1 release (FIG. 6G) by monocytes in response to MUC1-ST in a Siglec-9 dependent manner, as determined by ELISA. FIG. 6H is a bar graph depicting secretion of PAI-1 by monocytes incubated with T47D cells and T47D cells engineered to carry ‘healthy’ extended Core-2 O-linked glycans, as determined by ELISA. FIG. 6I is a bar graph depicting nitric oxide release by monocytes after incubation with MUC1-ST in the presence or absence of an anti-Siglec-9 antibody.

FIG. 7 is a table listing factors released by monocytes or macrophages after treatment with MUC1-ST, clustered into functional groups. Numbers refer to fold change from untreated cells, and black indicates no change.

FIGS. 8A-8F demonstrate that MUC1-ST engagement of Siglec-9 during the differentiation of monocytes into inflammatory macrophages results in the generation of dysfunctional cells. FIGS. 8A and 8B show CD86 expression by LPS and IFNγ differentiated M-CSF macrophages in the presence or absence of MUC1-ST or the indicated antibodies. FIG. 8A depicts representative flow cytometry histograms where the solid arrow indicates the presence of either anti-Siglec-9 or anti-6Rα antibody and the dotted arrow shows the control, and FIG. 8B depicts bar graphs summarizing the data from multiple independent donors. FIG. 8C is a bar graph showing IL-12 p70 release from LPS and IFNγ differentiated M-CSF macrophages in the presence or absence of MUC1-ST or the indicated antibodies (n=3 independent donors). FIG. 8D is a bar graph showing the effects of MUC1-ST treated macrophages on the proliferation of CD8+ or CD4+ T cells. FIG. 8E is a bar graph showing the effects of MUC1-ST treated macrophages on expression of CD69 in CD8+ or CD4+ T cells, as measured by flow cytometry (n=3 independent donors). FIG. 8F depicts representative density plots showing the percentage of CD69+CD25+CD8+ T cells following co-culturing with autologous M-CSF macrophages treated with MUC1-ST and antibody as indicated (n=3 independent donors). Data shown are the mean and s.e.m. Wherever indicated, * corresponds to p<0.05, ** to p<0.01, and *** to p<0.001 using paired or unpaired t-test where appropriate.

FIGS. 9A-9F depict modulation of the differentiation of monocyte derived dendritic cells by MUC1-ST binding to Siglec-9. FIG. 9A is a schematic illustrating the treatment regime for the indicated experiments. FIGS. 9B and 9C are bar graphs depicting the effect of MUC1-ST treatment on amounts of the indicated cell surface markers for monocytes differentiated into immature dendritic cells (FIG. 9B) or mature dendritic cells (FIG. 9C). The graph summarizes normalized MFI for 6 independent donors. FIG. 9D depicts histograms showing the ability of anti-Siglec-9 (arrowed) or anti-IL-6Rα (arrowed) antibodies to rescue the MUC1-ST mediated down-regulation of CD86 (thick black) in immature and mature DCs as compared to control (dotted arrow). FIGS. 9E and 9F are bar graphs showing normalized CD86 amounts for immature dendritic cells (FIG. 9E) or mature dendritic cells (FIG. 9F) after treatment with MUC1-ST or the indicated antibodies (n=6). Wherever indicated, * corresponds to p<0.05, ** to p<0.01, and *** to p<0.001 using paired or unpaired t-test where appropriate.

FIGS. 10A-10H identify factors which are associated with tumor progression that are secreted from MUC1-ST educated monocyte-derived macrophages. FIG. 10A is a schematic illustration showing the treatment regime for the indicated experiments. FIGS. 10B-10D are bar graphs showing the effects of MUC1-ST treatment on M-CSF secretion (FIG. 10B), PAI-1 secretion (FIG. 10C), or EGF secretion (FIG. 10C) for monocyte-derived macrophages, as assayed by ELISA (n=3 independent donors). FIGS. 10E-10F are bar graphs depicting anti-Siglec-9 antibody mediated inhibition of MUC1-ST induced M-CSF secretion (FIG. 10E), PAI-1 secretion (FIG. 10F), or EGF secretion (FIG. 10G) (n=3 independent donors). FIG. 10H is a bar graph depicting PAI-1 secretion by T47D cells or T47D cells engineered to carry Core-2 glycans associated with normal glycosylation following incubation with macrophages (n=2 independent donors).

FIGS. 11A-11F show that MUC1-ST educated monocyte-derived macrophages differentiate into tumor associated macrophages (TAMs). FIG. 11A depicts histograms showing the expression of CD206, CD163 and PD-L1 as analyzed by flow cytometry for macrophages with or without MUC1-ST or anti-Siglec-9 antibody treatment. Numbers refer to % positive cells and numbers in brackets to MFI (n=2 independent donors). FIGS. 11B and 11C are bar graphs depicting IDO mRNA as measured by qRT-PCR for monocyte-derived macrophages differentiated with GM-CSF (FIG. 11B) or M-CSF (FIG. 11C) and treated with MUC1-ST or anti-Siglec-9 antibody as indicated. FIG. 11D is a bar graph showing the presence of kynurenine in the supernatant from MUC1-ST treated macrophages.

FIGS. 11E and 11F are bar graphs showing CD8+ T cell proliferation (FIG. 11E) or IFNγ secretion (FIG. 11F) following co-culture of CD8+ T cells with macrophages treated with MUC1-ST or anti-Siglec-9 antibody as indicated. Wherever indicated, * corresponds to p<0.05, ** to p<0.01, and *** to p<0.001 using paired or unpaired t-test where appropriate.

FIGS. 12A-12E show that MUC1-ST induces monocytes to differentiate into tumor associated macrophages (TAMs) through MEK/ERK activation. FIG. 12A depicts images of cells at 400× magnification after treatment of monocytes from PBMCs with DMSO or PD98059 in the presence of MUC1-ST or PBS. FIG. 12B is a bar graph of live macrophage cell counts after treatment of monocytes from PBMCs with DMSO or PD98059 in the presence of MUC1-ST or PBS. FIG. 12C is tabulated flow cytometry data showing the mean fluorescent intensity of TAM associated markers on monocytes from PBMCs incubated with MUC1-ST or PBS (n=3). FIG. 12D is a bar graph depicting 524 nm absorbance after cells were stained with eosin (n=2). FIG. 12E is a bar graph depicting fluorescent intensity after cells were stained with anti-human Collagen type I-FITC (n=2).

FIGS. 13A-13K demonstrate that MUC1-ST binding to myeloid cells via Siglec-9 does not activate SHP1/2 but surprisingly induces calcium flux leading to MEK/ERK activation. FIG. 13A is a bar graph showing phosphorylation of Siglec-9 in monocytes treated with MUC1-ST or cross-linked anti-Siglec-9 antibody as indicated, as determined by ELISA (n=3 independent donors). FIG. 13B depicts a phospho-immunoreceptor array showing phosphorylation of Siglec-9 in monocytes treated with the indicated MUC1 glycoform. Top spots are phospho-Siglec-9 and bottom spots are reference. FIG. 13C is a Western blot showing SHIP-1 and phospho-SHIP-1 in monocytes treated with MUC1-ST or cross-linked anti-Siglec-9 as indicated. FIG. 13D is a line graph showing a time course of calcium flux in monocytes treated with MUC1-ST or anti-Siglec-9 antibody as indicated, as assayed by an intracellular fluorescent calcium reporter (n=3 independent donors). FIG. 13E is a bar graph showing calcium flux in monocytes 60 seconds after treatment with MUC1-ST or anti-Siglec-9 antibody as indicated, as assayed by an intracellular fluorescent calcium reporter (n=3 independent donors). FIG. 13F is a bar graph showing calcium flux for monocytes co-cultured with T47D cells carrying sialylated Core-1 or normal Core-2 glycans. FIGS. 13G and 13H are bar graphs showing secretion of PAI-1 (FIG. 13G) or M-CSF (FIG. 13H) by monocytes following treatment with MUC1-ST, D98059 or verapamil as indicated (n=3 independent donors). FIGS. 13I and 13J are bar graphs showing secretion of PAI-1 (FIG. 13I) or M-CSF (FIG. 13J) by macrophages following treatment with MUC1-ST, D98059 or verapamil as indicated (n=3 independent donors). FIG. 13K is a bar graph showing proliferation of CD8+ T cells following incubation with macrophages treated with MUC1-ST or PD98059 as indicated (n=2 independent donors). Wherever indicated, * corresponds to p<0.05, ** to p<0.01, and *** to p<0.001 using paired or unpaired t-test where appropriate.

### DETAILED DESCRIPTION

The invention is based, in part, upon the discovery that cancer cells in a subject that express certain sialylated Core-1-MUC1 glycoproteins not expressed by normal epithelial cells can facilitate immune recruitment, tumor microenvironment remodeling and tumor growth via the engagement of Siglec-9 expressed on the surface of certain myeloid cells, for example, monocytes, macrophages, and neutrophils. The cancer cells expressing such sialylated Core-1-MUC1 glycoproteins, can, through the engagement of Siglec-9, educate the myeloid cells to release factors associated with tumor microenvironment remodeling and disease progression, and to induce tumor-associated macrophages (TAMs) showing increased expression levels of the immune checkpoint ligand PD-L1, IDO, CD163 and CD206. As a result, the cancer cells expressing such sialylated Core-1-MUC1 glycoproteins can not only evade the immune system of the host subject but can also induce the differentiation of monocytes and macrophages into pro-tumorigenic TAMs. It has been discovered that these pro-tumorigenic effects can be mitigated or reversed by inhibiting Siglec-9 activity in the monocytes and macrophages educated following exposure to a sialylated Core-1-MUC1 glycoprotein.

In one aspect, the invention provides a method of treating cancer in a subject, for example, a human subject, in need thereof. The method comprises administering to the subject an effective amount of an inhibitor of Siglec-9 activity thereby to treat the cancer in the subject where the cancer has been identified as comprising cancerous cells that express one or more sialylated Core-1-MUC1 glycoproteins. As a result, the subject suitable for such treatment is characterized or identified as having a cancer comprising cancerous cells that express one or more sialylated Core-1-MUC1 glycoproteins, for example, MUC1-ST, MUC1-diST, or a combination thereof either alone or in association with other MUC1 glycoproteins comprising different glycans such as Core-2 glycans. The glycoproteins may be secreted from the cancerous cells and/or expressed on the cell surface of the cancerous cells.

In another aspect, the invention provides a method of reducing PDL-1 or IDO expression in a monocyte, macrophage, or neutrophil that expresses Siglec-9 and is capable of binding a sialylated Core-1-MUC1 glycoprotein (for example, MUC1-ST, MUC1-diST, or a combination thereof either alone or in association with other MUC1 glycoproteins comprising different glycans such as Core-2 glycans), expressed by a cancerous cell, for example, a human cancerous cell. The method comprises contacting the monocyte, macrophage, or neutrophil with an inhibitor of Siglec-9 activity thereby to reduce PD-L1 or IDO expression in the monocyte, macrophage, or neutrophil. The glycoprotein may be secreted from the cancerous cell and/or expressed on the cell surface of the cancerous cell.

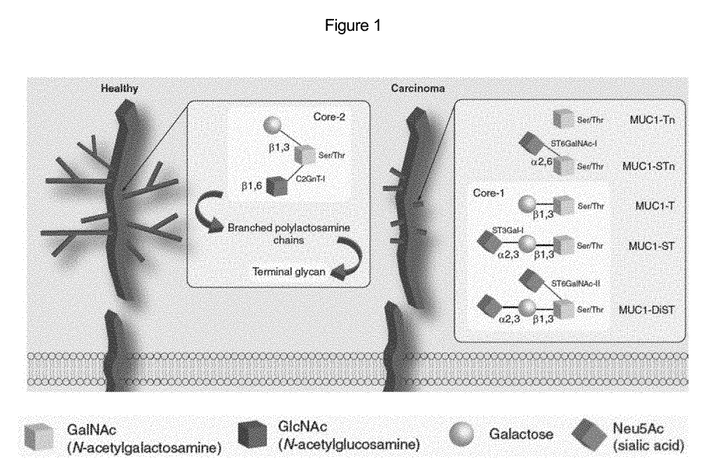

As used herein, the term “sialylated Core-1-MUC1 glycoprotein” refers to an O-linked glycosylated MUC1 protein, where the O-linked glycosylation comprises a sialylated Core-1 moiety linked to a serine or threonine amino acid in the MUC-1 protein. As used herein, the term “Core-1” is understood to mean a glycosyl group as shown in FIG. 1 and having the following structure:

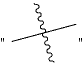

wherein

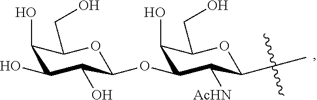

represents a covalent bond formed, for example, with a serine or threonine residue of MUC1. Exemplary sialylated Core-1-MUC1 glycoproteins include (i) MUC1-ST (NeuAcα2,3Galβ1-3GalNAc linked via a Ser/Thr of MUC1) as shown in FIG. 1 and having, for example, the following structure

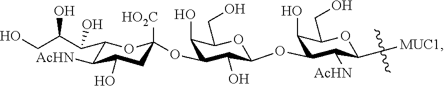

wherein

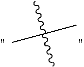

represents a covalent bond to a serine or threonine residue present in MUC1 and (ii) MUC1-DiST (NeuAcα2,3Galβ1-3[NeuAcα2,6]GalNAc linked via a Ser/Thr of MUC1) as shown in FIG. 1 and having, for example, the following structure

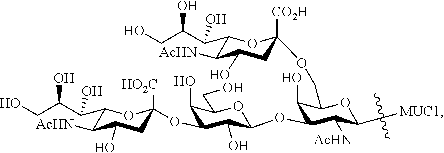

wherein

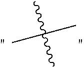

represents a covalent bond to a serine or threonine residue present in MUC1.

The sialylated Core-1-MUC1 glycoproteins are distinguishable from other sialylated glycoproteins, such as MUC1-STn, which is shown in FIG. 1 and having, for example, the following structure

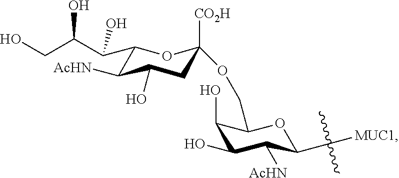

wherein

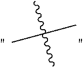

represents a covalent Dona to a serine or threonine residue present in MUC1, as well as other unsialylated Core-1 glycoproteins, such a MUC1-T, which is shown in FIG. 1 and having, for example the following structure

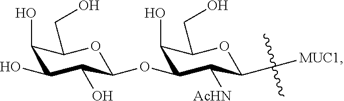

wherein

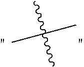

represents a covalent bond to a serine or threonine residue present in MUCL

As used herein, the term “MUC1” is understood to mean a protein comprising at least 5 consecutive repeats of the amino acid sequence of SEQ ID NO.: 1, for example, 5 to 200, 10 to 150, 10 to 100, 10 to 50, 15 to 150, 15 to 100, 15 to 50, 20 to 200, 20 to 100, 20 to 50, 25 to 200, 25 to 150, 25 to 100 or 25 to 50 consecutive repeats, or a protein comprising at least 5 consecutive repeats of an amino acid sequence having greater than 85%, 90%, 95%, 96%, 97%, 98% or 99% identity with SEQ ID NO.: 1, for example, 5 to 200, 10 to 150, 10 to 100, 10 to 50, 15 to 150, 15 to 100, 15 to 50, 20 to 200, 20 to 100, 20 to 50, 25 to 200, 25-150, 25 to 100 or 25 to 50 consecutive repeats. An exemplary amino acid sequence of a MUC1 protein comprises the amino acid sequence of SEQ ID NO.: 2, which comprises 33 consecutive repeats of the amino acid sequence of SEQ ID NO.: 1.

As used herein, the term “Siglec-9” is understood to mean a protein comprising the amino acid sequence of SEQ ID NO. 3, or comprising an amino acid sequence having greater than 85%, 90%, 95%, 96%, 97%, 98% or 99% identity with SEQ ID NO.: 3, or a fragment of any of the forgoing that is capable of binding to a sialylated Core-1 moiety, such as the sialylated Core-1 moiety of MUC1-ST. An exemplary amino acid sequence of Siglec-9 comprises SEQ ID NO: 4.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

As used herein, the term “primary monocyte” or “primary macrophage” is understood to mean a monocyte or macrophage that is isolatable or has been isolated from a subject, e.g., from blood or tissue of a subject. “Primary monocyte-derived macrophage” is understood to mean macrophages that can be obtained by culturing primary monocytes in vitro for at least 7 days in the presence of macrophage colony-stimulating factor (M-CSF) or granulocyte-macrophage colony-stimulating factor (GM-CSF).

### I. Inhibitors of Siglec-9 Activity

It is contemplated that a variety of inhibitors of Siglec-9 activity can be used in the practice of the invention. The inhibitors can completely or partially inhibit or otherwise reduce a given Siglec-9 activity or a given Siglec-9 mediated activity relative to an untreated control sample (e.g., a tissue or body fluid sample) or subject. For example, the inhibitor can be any agent that reduces sialylated Core-1-MUC1 glycoprotein (e.g., MUC1-ST and/or MUC1-DiST) induced activity of Siglec-9. For example, it is understood that certain inhibitors of Siglec-9 activity may act by blocking, reducing or otherwise neutralizing binding between sialylated Core-1-MUC1 glycoprotein (e.g., MUC1-ST and/or MUC1-DiST) and Siglec-9. In certain embodiments, the inhibitor binds to Siglec-9 to block, reduce or otherwise neutralize binding between sialylated Core-1-MUC1 glycoprotein (e.g., MUC1-ST and/or MUC1-DiST) and Siglec-9. In certain embodiments, the inhibitor binds to MUC1-ST to block, reduce or otherwise neutralize binding between sialylated Core-1-MUC1 glycoprotein (e.g., MUC1-ST and/or MUC1-DiST) and Siglec-9. Alternatively or in addition, the inhibitor of Siglec-9 activity may act by reducing the expression of Siglec-9 or the sialylated Core-1-MUC1 glycoprotein (e.g., MUC1-ST and/or MUC1-DiST), or by reducing the MUC1 glycosylation required for Siglec-9 binding. For example, a Siglec-9 inhibitor may target the sialyltransferase ST3Gal-I, which is responsible for the addition of the sialic acid to the Core-1 glycan forming ST. This enzyme is expressed by many normal cells in the haematopoietic system. It is over expressed compared to normal epithelial cells in breast and other carcinomas. Alternatively or in addition, the inhibitor of Siglec-9 activity, directly or indirectly, may inhibit the downstream effects of the interaction between MUC1-ST and Siglec-9 (e.g. calcium flux and/or MEK/ERK activation).

In certain embodiments, the inhibitor prevents differentiation of a macrophage into a tumor-associated macrophage (TAM). The inhibitor may induce the macrophage to differentiate into a pro-inflammatory macrophage and/or may prevent the loss of pro-inflammatory activity and/or may prevent the differentiation of a macrophage into a pro-tumorigenic, anti-inflammatory macrophage. The inhibitor may reduce upregulation of PD-L1, IDO, CD163, and CD206 expression in the myeloid cell educated by exposure to the sialylated Core-1-MUC1 glycoprotein.

Exemplary inhibitors of Siglec-9 activity include antibodies, nucleic acid-based therapeutics, such as aptamers and spiegelmers that bind to a target of interest, such as Siglec-9, or antisense or siRNAs molecules or CRISPR-Cas9 systems that inhibit expression and/or activity of a target of interest, such as Siglec-9, or small molecule inhibitors, for example, small molecule inhibitors of Siglec-9, MEK/ERK inhibitors or calcium flux inhibitors, or a combination thereof.

It is understood that, in certain embodiments, different inhibitors of Siglec-9 activity or different types of inhibitors of Siglec-9 activity may be administered in combination. For example, an inhibitor which acts by blocking, reducing or otherwise neutralizing binding between sialylated Core-1-MUC1 glycoprotein and Siglec-9 may be used in combination with an inhibitor which acts by inhibiting the downstream effects of the interaction between MUC1-ST and Siglec-9 (e.g. calcium flux and/or MEK/ERK activation).

### A. Protein-Based Therapeutics

In some embodiments, the inhibitor of Siglec-9 activity is a protein-based therapeutic. For example, in certain embodiments, the inhibitor of Siglec-9 activity is (i) an anti-Siglec-9 antibody, for example, a neutralizing anti-Siglec-9 antibody that prevents of reduces the binding of Siglec-9 to a sialylated Core-1-MUC1 glycoprotein (e.g., MUC1-ST and/or MUC1-DiST) or (ii) an anti-sialylated Core-1-MUC1 glycoprotein (e.g., MUC1-ST and/or MUC1-DiST) antibody, for example, a neutralizing antibody, that prevents or reduces the binding of sialylated Core-1-MUC1 glycoprotein (e.g., MUC1-ST and/or MUC1-DiST) to Siglec-9.

In certain embodiments, the antibody chosen acts to prevent the binding of the sialylated Core-1-MUC1 glycoprotein (e.g., MUC1-ST and/or MUC1-DiST) expressed by the cancerous cells to Siglec-9 expressed by a monocyte, macrophage, or neutrophil.

As used herein, unless otherwise indicated, the term “antibody” is understood to mean an intact antibody (e.g., an intact monoclonal antibody) or antigen-binding fragment of an antibody (e.g., an antigen-binding fragment of a monoclonal antibody), including an intact antibody or antigen-binding fragment that has been modified, engineered, or chemically conjugated. Examples of antibodies that have been modified or engineered include chimeric antibodies, humanized antibodies, and multispecific antibodies (e.g., bispecific antibodies). Examples of antigen-binding fragments include Fab, Fab′, (Fab′)₂, Fv, single chain antibodies (e.g., scFv), minibodies, and diabodies. In certain embodiments, an antibody, e.g., an anti-Siglec-9 antibody, is an antigen-binding fragment, e.g., a Fab, Fab′, (Fab′)₂, Fv, single chain antibody (e.g., scFv), minibody, or diabody. In certain embodiments, an antibody, e.g., an anti-Siglec-9 antibody, is a Fab. An example of a chemically conjugated antibody is an antibody conjugated to a toxin moiety.

In certain embodiments, the antibody binds to its target, for example, Siglec9, with a K_{D} of about 300 pM, 250 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, or 10 pM, or lower. In certain embodiments, the inhibitor is an anti-Siglec-9 neutralizing antibody, for example, having a binding affinity stronger than 1 nM for Siglec-9, for example, having a binding affinity lower than 1 nM.

The antibody may have a human IgG1, IgG2, IgG3, IgG4, or IgE isotype. In certain embodiments, the antibody has a human IgG4 isotype or another isotype that elicits little or no antibody-dependent cell-mediated cytotoxicity (ADCC) and/or complement mediated cytotoxicity (CDC). In certain embodiments, the antibody has a human an IgG4 isotype. In certain embodiments, the antibody has a human IgG1 isotype or another isotype that elicits antibody-dependent cell-mediated cytotoxicity (ADCC) and/or complement mediated cytotoxicity (CDC). In certain embodiments, the antibody has a human IgG1 isotype.

Exemplary anti-Siglec-9 antibodies are described in U.S. Pat. Nos. 8,394,382 and 9,265,826. Furthermore, exemplary anti-Siglec-9 antibodies include MAB1139 (Clone #191240, mouse IgG2a monoclonal) available from R&D Systems, Inc., AF1139 (Goat IgG polyclonal), available from R&D Systems, Inc., D18 (Sc-34936, Goat IgG polyclonal), available from Santa Cruz Biotechnology, Inc., Y-12 SC34938 (SC3-4938, goat IgG polyclonal), available from Santa Cruz Biotechnology, Inc., AB197981 (rabbit IgG polyclonal), available from Abcam, AB96545 (rabbit IgG polyclonal), available from Abcam, AB89484 (Clone # MM0552-6K12 mouse IgG2 monoclonal), available from Abcam, AB130493 (rabbit IgG polyclonal), available from Abcam, and AB117859 (Clone #3G8 mouse IgG1 monoclonal), available from Abcam.

Exemplary anti-MUC1 antibodies include MAB6298 (Clone #604804, IgG2b monoclonal), available from R&D Systems, Inc., AF6298 (Sheep IgG polyclonal), available from R&D Systems, Inc., HMFG2 (available from Ximbio), SM3 (Mouse IgG1 monoclonal, available from Abcam), KL-6 (available from EIDIA Co., Ltd. (Japan)) and MY.1 E12 (available from Professor Irimura, Department of Cancer Biology and Molecular Immunology, Faculty of Pharmaceutical Sciences, The University of Tokyo, Tokyo).

Methods for producing antibodies, for example, those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs encoding the desired antibodies. Production of defined gene constructs is within routine skill in the art.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L}, or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

A monoclonal antibody, for example, a monoclonal antibody that binds Siglec-9, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody, such as an anti-Siglec-9 antibody, are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains are said to be produced. See, e.g., U.S. Pat. No. 6,706,477 (Zauderer); U.S. Pat. No. 6,800,442 (Zauderer); and U.S. Pat. No. 6,872,518 (Zauderer).

Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

Additional exemplary protein-based therapeutics include soluble forms of the Siglec-9 extracellular domains. Such soluble receptor decoys could be used to sequester Siglec-9 ligands (such as MUC1-ST), and inhibit endogenous Siglec-9 activity. In one embodiment, the soluble Siglec-9 moiety comprises the sialic acid binding V-set immunoglobulin domain of Siglec-9 e.g., the soluble Siglec-9 moiety comprises SEQ ID NO: 3. In another embodiment, the soluble Siglec-9 moiety comprises extracellular domain of Siglec-9 e.g., the soluble Siglec-9 moiety comprises residues 1-326 of SEQ ID NO: 4. An exemplary soluble Siglec-9 moiety includes 1139-SL (a human Siglec-9 Fc chimera) available from R&D Systems, Inc.

### B. Nucleic Acid-Based Therapeutics

In addition, it is contemplated that inhibitors of Siglec-9 activity include nucleic acid-based therapeutics. It is understood that a nucleic acid-based therapeutic may include in addition to a nucleic acid component a non-nucleic acid component, for example, a protein component. Exemplary nucleic acid-based inhibitors of Siglec-9 activity include, for example, molecules that mimic antibody binding activity, for example, aptamers and spiegelmers, or antisense, siRNA, or snRNA molecules or CRISPR-Cas9 systems that modulate the expression and/or activity of a target molecule, such as Siglec-9.

Under certain circumstances, it may be desirable to use a binding moiety other than an antibody as an inhibitor of Siglec-9 activity. Exemplary nucleic acid based binding moieties include aptamers and spiegelmers. Aptamers are nucleic acid-based sequences that have strong binding activity for a specific target molecule. Spiegelmers are similar to aptamers with regard to binding affinities and functionality but have a structure that prevents enzymatic degradation, which is achieved by using nuclease resistant L-oligonucleotides rather than naturally occurring, nuclease sensitive D-oligonucleotides.

Aptamers are specific nucleic acid sequences that bind to target molecules with high affinity and specificity and are identified by a method commonly known as Selective Evolution of Ligands by Evolution (SELEX), as described, for example, in U.S. Pat. Nos. 5,475,096 and 5,270,163. Each SELEX-identified nucleic acid ligand is a specific ligand of a given target compound or molecule. The SELEX process is based on the observation that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets, which could include, for example, Siglec-9 or a Siglec-9 binding cognate sialylated Core-1 MUC1 glycoprotein (for example, MUC1-ST).

The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule. Thus, this method allows for the screening of large random pools of nucleic acid molecules for a particular functionality, such as binding to a given target molecule.

The SELEX method also encompasses the identification of high-affinity nucleic acid ligands containing modified nucleotides conferring improved characteristics on the ligand, such as improved in vivo stability and protease resistance. Examples of such modifications include chemical substitutions at the ribose and/or phosphate and/or base positions. SELEX process-identified nucleic acid ligands containing modified nucleotides are described in U.S. Pat. Nos. 5,660,985 and 5,580,737, which include highly specific nucleic acid ligands containing one or more nucleotides modified at the 2′ position with, for example, a 2′-amino, 2′-fluoro, and/or 2′-O-methyl moiety. For example, aptamers to MUC-1 are described, for example, in U.S. Pat. No. 8,129,506 and Hu et al. (2012) PLOS ONE 7(2):e31970. It is contemplated that the skilled person can develop aptamers using conventional technologies that specifically bind, for example, Siglec-9 or a sialylated Core-1-MUC1 glycoprotein, such as MUC1-ST, for use in the practice of the invention (see, Yang, et al. (2014) J. HEMATOL. ONCOL. 7:5).

Instead of using aptamers, which may require additional modifications to become more resistant to nuclease activity, it is contemplated that spiegelmers, mirror image aptamers composed of L-ribose or L-2′deoxyribose units (see, U.S. Pat. Nos. 8,841,431, 8,691,784, 8,367,629, 8,193,159 and 8,314,223) can be used in the practice of the invention. The chiral inversion in spiegelmers results in an improved plasma stability compared with natural D-oligonucleotide aptamers. L-nucleic acids are enantiomers of naturally occurring D-nucleic acids that are not very stable in aqueous solutions and in biological systems or biological samples due to the widespread presence of nucleases. Naturally occurring nucleases, particularly nucleases from animal cells are not capable of degrading L-nucleic acids. Because of this, the biological half-life of the L-nucleic acid is significantly increased in such a system, including the animal and human body. Due to the lacking degradability of L-nucleic acids, no nuclease degradation products are generated and thus no side effects arising therefrom observed.

Using in vitro selection, an oligonucleotide that binds to the synthetic enantiomer of a target molecule, e.g., a D-peptide, can be selected. The resulting aptamer is then resynthesized in the L-configuration to create a spiegelmer (from the German “spiegel” for mirror) that binds the physiological target with the same affinity and specificity as the original aptamer to the mirror-image target. This approach has been used to synthesize spiegelmers that bind, for example, hepcidin (see, U.S. Pat. No. 8,841,431), MCP-1 (see, U.S. Pat. Nos. 8,691,784, 8,367,629 and 8,193,159) and SDF-1 (see, U.S. Pat. No. 8,314,223). It is contemplated that the skilled person could develop spiegelmers using conventional technologies that specifically bind, for example, Siglec-9 or a sialylated Core-1-MUC1 glycoprotein, such as MUC1-ST, for use in the practice of the invention.

In addition, it is contemplated that other useful nucleic acid-based therapeutics can include, for example, antisense or siRNA molecules or CRISPR-Cas9 systems that modulate the expression and/or activity of a target molecule, such as Siglec-9. Exemplary siRNA antisense molecules that are inhibitors of Siglec 9 activity include, for example, sc-106550, available from Santa Cruz Biotechnology, Inc. Exemplary shRNA antisense molecules that are inhibitors of Siglec 9 activity include, for example, sc-106550-SH, available from Santa Cruz Biotechnology, Inc. Exemplary CRISPR-Cas9 systems that are inhibitors of Siglec 9 activity include, for example, pre-designed Siglec 9 targeting single guide RNAs such as GSGH11838-246555148, GSGH11838-246555148, or GSGH11838-246555153, used in conjunction with the Cas9 nuclease, for example, CAS10136, available from GE Dharmacon.

### C. Small Molecule-Based Therapeutics

In addition, it is contemplated that inhibitors of Siglec-9 activity include small molecule-based therapeutics. Exemplary small molecule inhibitors of Siglec-9 activity include sialic acid mimetics that target Siglec-9 (see Büll et al. (2016) TRENDS BIOCHEM. SCI. 41(6): 519-31, which describes the Siglec-9 compound referred to as CD329; Rillahan et al. (2012) ANGEW. CHEM. INT. Ed. ENGL, 51:11014).

In addition, it is possible that the inhibitors may inhibit the downstream effects of the interaction between MUC1-ST and Siglec-9 (e.g., calcium flux and/or MEK/ERK activation). Exemplary MEK/ERK inhibitors are described in U.S. Pat. Nos. 7,378,423, 8,580,304, 8,703,781, 8,835,443, 9,155,706, and 9,271,941 and include the small molecule trametinib (GlaxoSmithKline, LLC). Exemplary inhibitors of calcium flux are described in Elliot et al., (2011) J. CLIN. HYPERTENS. 13(9): 687-9, and include the small molecules verapamil, diltiazem, nifedipine, nicardipine, isradipine, felodipine, amlodipine, nisoldipine, clevidipine, and nimodipine.

### II. Pharmaceutical Compositions, Methods of Administration, and Therapeutic Uses

The methods and compositions disclosed herein can be used to treat a variety of cancers and cancerous conditions, where the cancer comprises cancerous cells that express one or more sialylated Core-1-MUC1 glycoproteins. These may include, but are not limited to, blood-based cancers (e.g., chronic myelogenous leukemia, chronic myelomonocytic leukemia, Philadelphia chromosome positive acute lymphoblastic leukemia, mantle cell lymphoma), prostate cancer, gastric cancer, colorectal cancer, skin cancer (e.g., melanomas or basal cell carcinomas), lung cancer (e.g., non-small cell lung cancer), breast cancer, cancers of the head and neck, bronchus cancer, pancreatic cancer, urinary bladder cancer, cancers of the brain or central nervous system, peripheral nervous system cancer, esophageal cancer, cancer of the oral cavity or pharynx, liver cancer (e.g., hepatocellular carcinoma), kidney cancer (e.g., renal cell carcinoma), testicular cancer, biliary tract cancer, small bowel or appendix cancer, gastrointestinal stromal tumor, salivary gland cancer, thyroid gland cancer, adrenal gland cancer, osteosarcoma, chondrosarcoma, cancer of hematological tissues, and the like.

Cancer or cancerous cells can be in the form of a tumor (i.e., a solid tumor), exist alone within a subject (e.g., leukemia cells), or be cell lines derived from a cancer.

In certain embodiments, the methods disclosed herein can be used to treat breast (e.g., Luminal A, Luminal B, Basal-like, Her2-enriched, and normal-like breast cancer), colon, lung, ovarian, pancreatic or prostate cancer. Furthermore, the cancer may be an adenocarcinoma. Furthermore, the cancer may be a metastatic cancer and/or a refractory cancer.

The inhibitors of Siglec-9 activity should be formulated, for example, with a pharmaceutically acceptable carrier, suitable for administration to a subject in need of treatment. As used herein, the term “pharmaceutically acceptable carrier” is understood to mean one or more of a buffer, carrier, or excipient suitable for administration to a subject, for example, a human subject, without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

As used herein, the terms “treat,” “treating” and “treatment” is understood to mean any effect, e.g., lessening, reducing, modulating, ameliorating, or eliminating, that results in the improvement of the condition, disease, disorder, and the like, or ameliorating a symptom thereof. As used herein, an “effective amount” of an inhibitor of Siglec-9 activity refers to the amount of such an agent sufficient to effect beneficial or desired results including treating, alleviating, ameliorating, relieving, delaying onset of, inhibiting progression of, reducing severity of, and/or reducing incidence of one or more symptoms or features of cancer.

Pharmaceutical compositions containing therapeutic agents, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, subcutaneous, intratumoral, intrapleural, and rectal administration. A preferred route of administration for antibody-based therapeutics is via IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

Generally, a therapeutically effective amount of an active component (e.g., an antibody) is in the range of 0.1 mg/kg to 100 mg/kg, e.g., 1 mg/kg to 100 mg/kg, e.g., 1 mg/kg to 10 mg/kg, e.g., 2.0 mg/kg to 10 mg/kg. The amount administered will depend on variables such as the type and extent of disease or indication to be treated, the overall health of the patient, the in vivo potency of the therapeutic agent, the pharmaceutical formulation, the serum half-life of the therapeutic agent, and the route of administration.

The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody or fusion protein, and the disease being treated. Exemplary dosing frequencies are once per day, once per week and once every two weeks. In some embodiments, dosing is once every two weeks.

In certain embodiments, the administration of the therapeutic agent (for example, antibody-based therapeutics) is by parenteral administration, e.g., IV infusion. In some embodiments, the therapeutic agents are lyophilized, and then reconstituted in buffered saline, at the time of administration. The effective amount of a second therapeutic agent, for example, an anti-cancer agent or the other agents discussed below, will also follow the principles discussed hereinabove and will be chosen so as to elicit the required therapeutic benefit in the patient.

### III. Combination Therapies

Given that cancer cells expressing sialylated Core-1-MUC1 glycoproteins, can, through the engagement of Siglec-9, induce the differentiation of myeloid cells into tumor-associated macrophages (TAMs) showing increased expression levels of the immune checkpoint ligand PD-L1 and IDO, it is contemplated that the inhibitor of Siglec-9 activity can be administered together (either simultaneously or sequentially) with an IDO inhibitor and/or or an immune checkpoint inhibitor, for example, a PD-1 inhibitor, PD-L1 inhibitor, CTLA-4 inhibitor, adenosine A_{2A} receptor inhibitor, B7-H3 inhibitor, B7-H4 inhibitor, BTLA inhibitor, KIR inhibitor, LAG3 inhibitor, TIM-3 inhibitor, VISTA inhibitor or TIGIT inhibitor.

IDO is the first and rate-limiting enzyme in the tryptophan metabolic pathway, and is overexpressed by many cancer cells. IDO overexpression leads to a local depletion of tryptophan and a subsequent amino acid starvation response in cytotoxic T-cells. Furthermore, tryptophan metabolites that result from IDO activity activate regulatory T-cells, further dampening the immune response. Accordingly, in one embodiment the inhibitor of Siglec-9 activity is administered together with (either together or sequentially) an IDO inhibitor. Exemplary IDO inhibitors are described in U.S. Pat. Nos. 8,034,953, 8,088,803, 8,232,313, 8,389,568 and PCT Publication No. WO2014/150677, and include the small molecules INCB024360 (Incyte Corporation), Indoximod (NewLink Genetics), NLG919 (NewLink Genetics), and F001287 (Flexus Biosciences).

A number of T-cell checkpoint inhibitor pathways have been identified to date, for example, the PD-1 immune checkpoint pathway and Cytotoxic T-lymphocyte antigen-4 (CTLA-4) immune checkpoint pathway. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, PD-L2, etc., that interact with PD-1 on the surface of T-cells to shut down or modulate T-cell activity. Using this approach, cancer can evade the T-cell mediated immune response.

In certain embodiments, the immune checkpoint inhibitor prevents (completely or partially) an antigen expressed by the cancerous cell from repressing T-cell inhibitory signaling between the cancerous cell and the T-cell. In one embodiment the immune checkpoint inhibitor is mediated via a PD-1 mediated cascade. Examples of such immune checkpoint inhibitors include, for example, anti-PD-1 antibodies, anti-PD-L1 antibodies, and anti-PD-L2 antibodies. Accordingly, in one embodiment the inhibitor of Siglec-9 activity is administered with a PD-1-based immune checkpoint inhibitor, which can include (1) a molecule (for example, an antibody or small molecule) that binds to a PD-1 ligand (for example, PD-L1 or PD-L2) to prevent the PD-1 ligand from binding to its cognate PD-1, and/or (2) a molecule (for example, an antibody or small molecule) that binds to PD-1 to prevent the PD-1 from binding of its cognate PD-1 ligand.

Exemplary PD-1/PD-L1 based immune checkpoint inhibitors include antibody based therapeutics and nucleic acid based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Bristol-Myers Squibb Co.), pembrolizumab (KEYTRUDA®, Merck & Co.), atezolizumab (formerly MPDL3280A), MEDI4736, avelumab, PDR001, pidilizumab (CT-011, Cure Tech) and BMS 936559 (Bristol Myers Squibb Co.). Exemplary anti-PD-L1 antibodies are described, for example, in U.S. Pat. Nos. 9,273,135, 7,943,743, 9,175,082, 8,741,295, 8,552,154, and 8,217,149.

Exemplary siRNAs for silencing PD-1 are available from ThermoFisher (Catalog No. AM16708. Additional exemplary siRNAs for silencing PD-1 are described in Iwamura (2012) NATURE GENE THERAPY 19: 959-966. Exemplary siRNAs for silencing PD-1 ligands are described in U.S. Pat. No. 9,181,525 and Breton et al. (2009) J. CLIN. IMMUNOL., 29(5): 637-645. Exemplary aptamers that inhibit the PD-1/PD-L1 axis are described in Prodeus et al., (2015) MOL. THER. NUCLEIC ACIDS 28:4 e237.

In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. In one embodiment, the immune checkpoint inhibitor is a CTLA-4 inhibitor. Examples of such immune checkpoint inhibitors include, for example, a molecule (for example, an antibody or small molecule) that binds to CTLA-4 on a T-cell to prevent the binding of a CTLA-4-ligand expressed by the cancer cell of interest. Other examples of such immune checkpoint inhibitors include nucleic acid-based inhibitors of CTLA-4 activity, for example, molecules that mimic antibody binding activity, for example, aptamers and spiegelmers, or antisense, siRNA, or snRNA molecules that modulate the expression and/or activity of CTLA-4. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, PCT Publication Nos. WO 98/42752, WO 00/37504, WO 01/14424, European Patent No. EP 1212422 B1. Exemplary CTLA-4 antibodies include ipilimumab or tremelimumab. Exemplary CTLA-4 inhibiting nucleic acids include CTLA-4 siRNA (for example, ThermoFisher Cat No. AM16708). Furthermore, CTLA-4 aptamers are described, for example, in Santulli-Marotto et al., (2003) CANCER RES. 63(21): 7483-9).

Additional exemplary immune checkpoint inhibitor targets include the adenosine A_{2A} receptor; B7-H3 (CD276), B7-H4 (VTCN1); B and T lymphocyte attenuator (BTLA, CD272); killer-cell immunoglobulin-like receptor (KIR); lymphocyte activation gene-3 (LAG3); and T-cell immunoglobulin domain and mucin domain-3 (TIM-3). Additional exemplary immune checkpoint inhibitor antibodies include the anti-B7H3 antibody enoblituzumab (MGA271, MacroGenics, Inc.), the anti-MR antibody lirilumab (Bristol-Myers Squibb Co.), the anti-LAG3 antibody BMS-986016 (Bristol-Myers Squibb Co), the anti-TIM-3 antibody RMT3-23 (Rat IgG2a monoclonal, available from BioLegend), and anti-B7-H4 scFvs described in Dangaj et al. (2015) METHODS MOL. BIOL. 1319: 37-49. Additional exemplary immune checkpoint inhibitor small molecules include the adenosine A_{2A} receptor antagonist SCH58261 (Mittal et al. (2014) CANCER RES. 74: 3652-8). Suitable VISTA inhibitors may include antibodies such as that described by Wang et al J. Exp Med 2011, 2018: 577-592. Similarly, TIGIT inhibitors may be antibodies as described for example by Johnston R J et al. Oncoimmunology 2015 May 27; 4(9).

### IV. Diagnostic Methods

In another aspect, the invention provides a method of identifying a subject with cancer likely to respond to treatment with an inhibitor of Siglec-9 activity. The method comprises determining whether the cancer comprises cancerous cells that express one or more sialylated Core-1-MUC1 glycoproteins (for example, MUC1-ST, MUC1-diST, or a combination thereof). It is contemplated that a variety of detection methods can be used in the practice of the invention.

A variety of samples, for example, a tissue sample, such as tumor tissue, or body fluid sample, such as whole blood, serum, plasma, urine, etc. may be used in such a diagnostic method. By way of example, a tissue sample from a tumor in a human subject (e.g., a tissue sample from a tumor harvested from a human subject, e.g., a human subject being considered for treatment with a Siglec-9 inhibitor) can be used as a source of protein, or a source of thin sections for immunohistochemistry (IHC), so the existence and/or level of sialylated Core-1-MUC1 glycoproteins in the sample can be determined in practicing the disclosed methods. The tissue sample can be obtained by using conventional tumor biopsy instruments and procedures. Endoscopic biopsy, excisional biopsy, incisional biopsy, fine needle biopsy, punch biopsy, shave biopsy and skin biopsy are examples of recognized medical procedures that can be used by one of skill in the art to obtain tumor samples. The tumor tissue sample should be large enough to provide sufficient protein, or thin sections for detecting and/or measuring the levels of sialylated Core-1-MUC1 glycoproteins.

The sample can be in any form that allows measurement of sialylated Core-1-MUC1 glycoprotein content. In other words, the sample must be sufficient for protein extraction, or processing to permit detection of the Core-1-MUC1 glycoprotein, such as, preparation of thin sections. Accordingly, the sample can be fresh, preserved through suitable cryogenic techniques, or preserved through non-cryogenic techniques. A standard process for handling clinical biopsy tissue specimens is to fix the tissue sample in formalin and then embed the sample in paraffin. Samples in this form are commonly known as formalin-fixed, paraffin-embedded (FFPE) tissue. Suitable techniques of tissue preparation for subsequent analysis are well-known to those of skill in the art, but the use of FFPE sections would be particularly useful for looking for MUC1-ST expression.

The presence and level of sialylated Core-1-MUC1 glycoproteins in a tumor sample, or clinical specimen, can be determined (e.g., visualized) by immunohistochemistry (IHC) or immunofluorescence (IF). Because clinical specimens often are preserved as formalin fixed paraffin embedded (FFPE) blocks, IHC and IF are particularly useful for measuring sialylated Core-1-MUC1 glycoproteins in clinical specimens. Assaying sialylated Core-1-MUC1 glycoproteins by IHC or IF uses at least one antibody that can bind sialylated Core-1-MUC1 glycoproteins (the detection antibody). Using standard techniques, the antibody can be used to detect the presence of sialylated Core-1-MUC1 glycoproteins in thin sections, e.g., 5 micron sections, obtained from tumors, including FFPE sections and frozen tumor sections. Typically, the tumor sections are initially treated in such a way as to retrieve the antigenic structure of proteins that were fixed in the initial process of collecting and preserving the tumor material. Slides are then blocked to prevent non-specific binding by the detection antibody. The presence and/or amount of sialylated Core-1-MUC1 glycoproteins is then detected by using the detection antibody and a secondary antibody. The secondary antibody, which recognizes and binds to the detection antibody, is linked to an enzyme or fluorophore. Typically, the tumor sections are washed and blocked with non-specific protein such as bovine serum albumin between steps. If the secondary antibody is linked to an enzyme, the slide is developed using an appropriate enzyme substrate to produce a visible signal. If the secondary antibody is linked to a fluorophore, the slide is viewed by using a fluorescence microscope. The samples can be counterstained with haematoxylin.

The presence and/or level of sialylated Core-1-MUC1 glycoproteins can also be determined by an enzyme linked immunosorbent assay (ELISA). Performing an ELISA uses at least one antibody capable of binding sialylated Core-1-MUC1 glycoproteins (the detection antibody). Sialylated Core-1-MUC1 glycoprotein (e.g., glycoprotein expressed on a cell surface or free) in a sample to be analyzed can be immobilized on a solid support such as a polystyrene microtiter plate. This immobilization can be by non-specific binding, i.e., through adsorption to the surface. Alternatively, immobilization can be by specific binding, i.e., through binding by a capture antibody (e.g., via an antibody that binds sialylated Core-1-MUC1 glycoprotein that is different from the detection antibody), in a “sandwich” ELISA. After the protein is immobilized, the detection antibody is added, and the detection antibody forms a complex with the immobilized sialylated Core-1-MUC1 glycoprotein. The detection antibody is linked to an enzyme, either directly or indirectly, e.g., through a secondary antibody that specifically recognizes the detection antibody. Typically between each step, the plate, with bound sialylated Core-1-MUC1 glycoproteins, is washed with a mild detergent solution. Typical ELISA protocols also include one or more blocking steps, which involve use of a non-specifically-binding protein such as bovine serum albumin to block unwanted non-specific binding of protein reagents to the plate. After a final wash step, the plate is developed by addition of an appropriate enzyme substrate to produce a visible signal, which indicates the quantity of sialylated Core-1-MUC1 glycoprotein in the sample. The substrate can be, e.g., a chromogenic substrate or a fluorogenic substrate. ELISA methods, reagents and equipment are well-known in the art and commercially available.

The foregoing approaches, for example, immunohistochemistry (IHC), immunofluorescence (IF), or ELISA may be performed directly with a detection antibody that specifically binds a sialylated Core-1-MUC1 glycoprotein. Alternatively, it is possible to detect and/or measure the amount of sialylated Core-1-MUC1 glycoprotein, for example, MUC1-ST, without using an antibody that binds to the sialic acid moiety of the glycoprotein, for example, an antibody that can only bind non-sialylated Core-1-MUC1 glycoproteins. In such an approach, the foregoing or any other antibody based detection methods may be performed by using an antibody specific for non-sialylated Core-1-MUC1 glycoproteins, where binding and quantification are determined before and after treatment with a neuraminidase enzyme. The neuraminidase enzyme removes the sialic acid moiety, and the difference in signal before and after neuraminidase treatment can be attributed to the sialylated Core-1-MUC1 glycoproteins. An example of such an indirect method using a neuraminidase enzyme treatment step is described in Example 1.

Once a subject has been identified as likely to respond to treatment with an inhibitor or Siglec-9 activity, the subject may be treated with one or more inhibitors of Siglec-activity, such as one or more of the inhibitors described herein above, such as an anti-Siglec-9 antibody that prevents or otherwise reduces the binding of Siglec-9 and its cognate ligand, namely, the Core-1-MUC1 glycoprotein, so as to treat the cancer.

Throughout the description, where apparatus, devices, and systems are described as having, including, or comprising specific components, or where processes and methods are described as having, including, or comprising specific steps, it is contemplated that, additionally, there are apparatus, devices, and systems of the present invention that consist essentially of, or consist of, the recited components, and that there are processes and methods according to the present invention that consist essentially of, or consist of, the recited processing steps.

Practice of the invention will be more fully understood from the foregoing examples, which are presented herein for illustrative purposes only, and should not be construed as limiting the invention in any way.

### EXAMPLES

### Materials and Methods

In general, the actual reagents and protocols used in each of the following Examples are set forth in the specific examples. However, unless indicated, T47D cells were cultured in RPMI 1640 (Lonza) supplemented with 100 units/mL penicillin, 100 μg/mL streptomycin, 2 mmol/L L-glutamine and 10% heat-inactivated FCS (all Life Technologies). T47D cells transfected with C2GNT1 as described in Dalziel et al. (2001) J. BIOL. CHEM. 276: 11007-11015 were additionally cultured with 500 ug/ml G418.

Both the cell phenotype staining and neutralization studies described in the Examples were performed using the following antibodies (all anti-human): Siglec-9 (Biotechne; 191240), Siglec-9 FITC (Biotechne; 191240), Siglec-3 (Biotechne; 6C5/2), Siglec-7 (Biotechne; AF1138), Siglec-3 FITC (BD; P67.6), Siglec-7 PE (Biolegend; 6-434), Siglec-10 PE (Biolegend; 5G6), Siglec-1 (abcam; 7D2), HLA-DR FITC (Beckman Coulter; IM0463U), CD16 FITC (Biolegend; B73.1), CD14 FITC (BD 555397), CD69 PE (Beckman Coulter; IM1943), CD25 FITC (ABX228FITC), CD86 PE (Beckman Coulter; IM2729U), CD40 FITC (BD 555588), CD83 PE (Beckman Coulter; IM2218), CD163 PE (Biolegend; GHI/61), CXCR1 FITC (BD Pharmongen; 551126), CD45 PC5 (Beckman Coulter; IM2653U), CD206 PE (ebioscience; 19.2), PD-L1 PE (Biolegend; 29E.2A3), CD36 (Santa Cruz; H-300), TGFbetaRII (Bio-Techne; AF-241), IL-6Rα (Tocilizumab; Roche. A generous gift from Dr. Valerie Corrigall). Cells were suspended in PBS+0.5% BSA (2×10⁵ cells/100 μL/sample) and incubated with Abs according to the manufacturer's instructions. At least 1×10⁴ events were evaluated using either Epics XL, (Beckman Coulter) or FACSCalibur (BD Biosciences) flow cytometers. Analysis was performed using either WinMDI or Cellquest software.

ELISAs for IL-6, IL-12p70, TGF-β1, PAI-1, M-CSF, EGF, SHP2, phospho-SHP2 (Biotechne) were all carried out as per manufacturer's instructions.

### Example 1—MUC1-ST Binds to Siglec-9 Expressed by Primary Monocytes and Macrophages

This example demonstrates that Siglec-9 expressed by primary monocytes and monocyte-derived macrophages binds to a form of MUC1 carrying short, sialylated Core-1 glycans (NeuAcα2,3Galβ1-3GalNAc) known as MUC1-ST, which is expressed by cancer cells.

To investigate the interaction of MUC1-ST with cells of the immune system, immune cell subsets were obtained as follows. Leukocyte reduction system (LRS) cones were purchased from the National Blood Transfusion Service (NBTS, Tooting, UK) and centrifuged on a Ficoll gradient (Ficoll-Paque PREMIUM, GE Healthcare) at 400×g. CD14+, CD19+, CD8+, CD4+ cells were isolated from PBMCs using microbeads (MACS system; Miltenyi Biotech) according to the manufacturer's instructions. Purity was assessed at >95% by staining with relevant antibodies.

To differentiate monocytes into macrophages, CD14+ cells were plated at a concentration of 1×10⁶/mL in AIM V medium (Lonza) with either 50 ng/mL recombinant human M-CSF or 50 ng/mL recombinant human GM-CSF (Bio-Techne). The cytokines were added every 3 days. The cells were incubated at 37° C., 5% CO₂ for 7 days to fully differentiate, before being characterized as macrophages via phenotypic flow cytometric analysis. To differentiate monocytes into dendritic cells (moDC), CD14+ cells were plated at a concentration of 1×10⁶/mL in AIM V medium with 1500 U/mL recombinant human IL-4 (Bio-Techne) and 400 U/mL human GM-CSF (Bio-Techne) for 6 days to fully differentiate, before being characterized as immature DCs via phenotypic flow cytometric analysis (Epics XL, Beckman Coulter or FACSCalibur, BD Biosciences plus WinMDI or Cellquest software). MoDCs were matured using 1 μg/mL LPS for 24 hours.

Recombinant tumor-associated MUC1 glycoforms were prepared as follows. Recombinant secreted MUC1 consisting of 16 tandem repeats carrying sialylated Core-1 and fused to mouse Ig was produced in CHO cells as previously described (Backstrom et al. (2003) BIOCHEM J. 376: 677-86; Link et al. (2004) J. BIOTECHNOL. 110: 51-62). Concentrated supernatant was treated with 10 mg trypsin per mg MUC1-ST-IgG for 2 hours (MUC1 tandem repeats are not sensitive to trypsin digestion) to remove the Ig. The treated supernatant was applied to a HiPrep 16/10 Q FF anion exchange column, which was washed to remove the unbound material with 20 column volumes of 50 mM Tris-HCl pH 8.0. The MUC1-ST was eluted as previously described (Backstrom et al. (2003) supra). The purity of the product was determined by a negative result in a mouse IgG ELISA, silver staining of SDS PAGE and amino acid composition. All batches of purified MUC1-ST were tested for lack of endotoxin using the LAL assay (Lonza) as per manufacturer's instructions, TGFβ using an ELISA (Bio-Techne) as per manufacturer's instructions, and protease activity using the casein cleavage assay (Pierce/ThermoFisher) as per manufacturer's instructions. The product was quantitated either by amino acid analysis (Alta Bioscience) or using an HMFG2:HMFG2 sandwich ELISA against a previously quantified batch. The endotoxin levels of MUC1-ST were 0.004-0.002 EU/μg, well below the limits required for immunological experiments.

MUC1 carrying Core-1 was produced by dialyzing purified MUC1-ST in 50 mM NaAc pH 6.0, 4 mM CaCl₂) overnight (O/N) at 4° C., and then treating with 0.15 U/mg neuraminidase (NA) on agarose beads (Sigma) O/N at RT and then dialysed against PBS O/N. Cleavage of sialic acids was measured by HMFG2:lectin ELISA. Briefly, 1 μg/mL HMFG2 in PBS was bound to plastic O/N, before being blocked (1% BSA in PBS) and the samples (pre and post NA treatment) were loaded and incubated at RT for 2 hours. Sugars were analysed using 1 μg/mL biotinylated PNA (which binds exposed galactose residues and does not bind ST) and 5 ug/mL biotinylated MAA (which binds alpha 2,3 linked sialic acids and does not bind T).

Unglycosylated MUC1 was produced in CHO 1d1D cells as previously described (Beatson et al. (2015) PLOS ONE 10:e0125994) without the addition of 1 mM GalNAc to the growth medium. Biotinylation of these glycoforms was performed as previously described (Beatson et al. (2015) supra).

Unless indicated otherwise, all binding experiments using purified immune cell subsets and biotinylated purified recombinant tumor-associated MUC1 glycoforms were performed as follows. 1×10⁵ isolated/differentiated cells at 5×10⁵ cells per mL were incubated for 4 hours on ice with 10 μg of the appropriate biotinylated recombinant MUC1 glycoform in 0.5% BSA in PBS. Cells were washed in 0.5% BSA in PBS before 1:200 SAPE (Life Technologies) was added for 30 minutes on ice. Cells were washed and analysed by flow cytometry or fluorescent microscopy (after cytospin), using streptavidin-PE (SAPE) as a label.

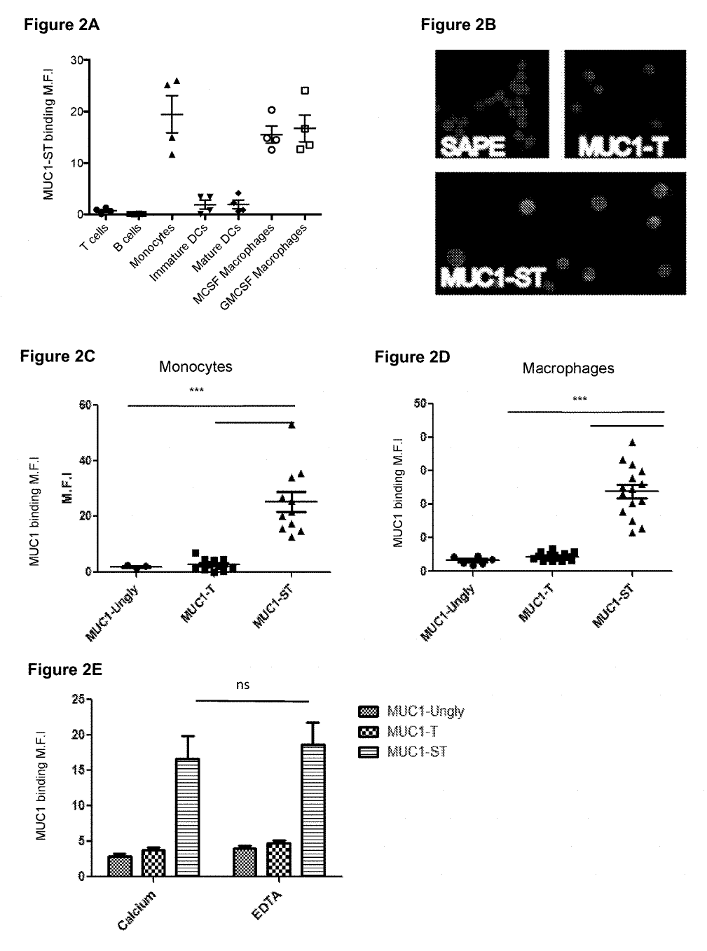

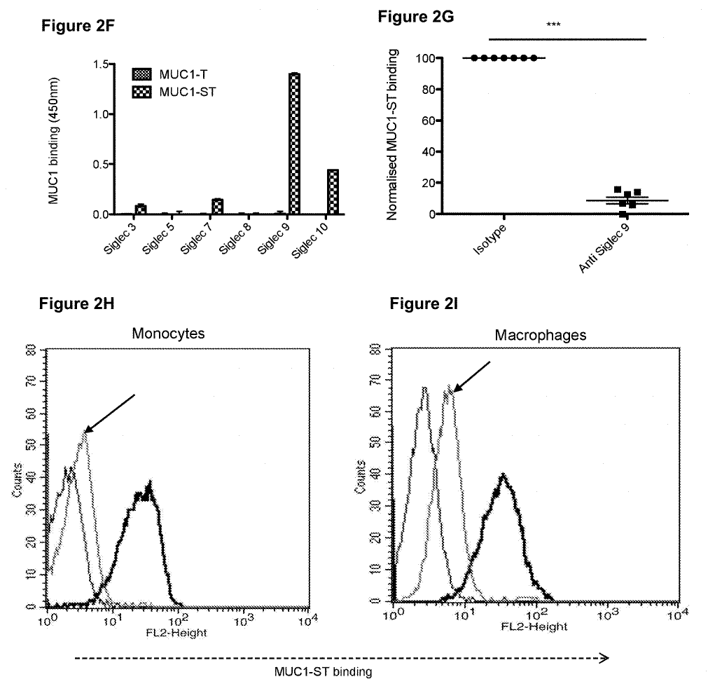

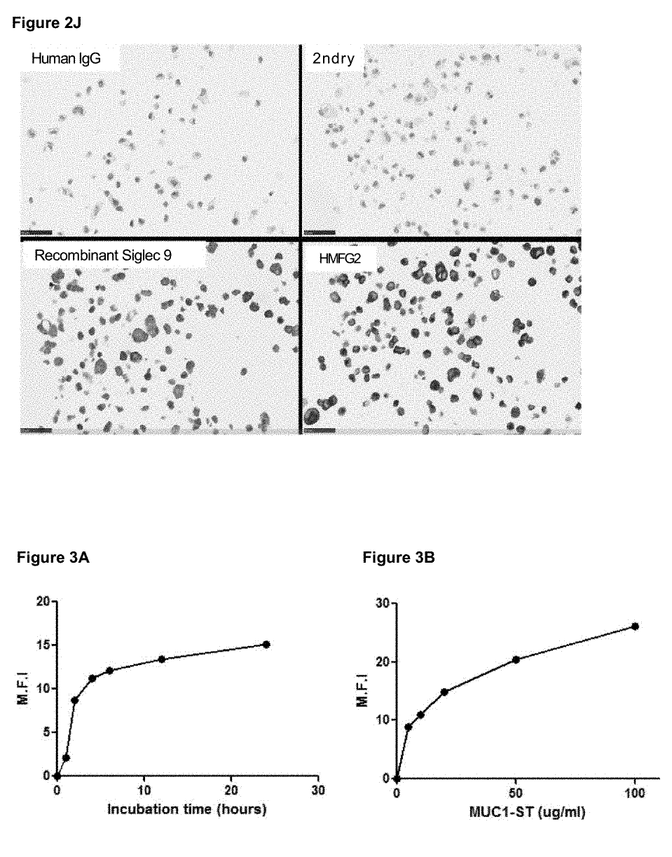

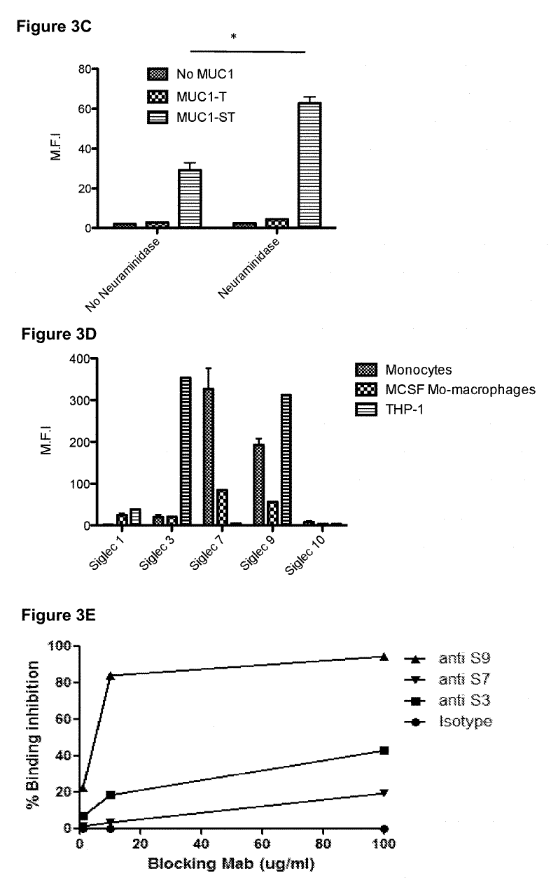

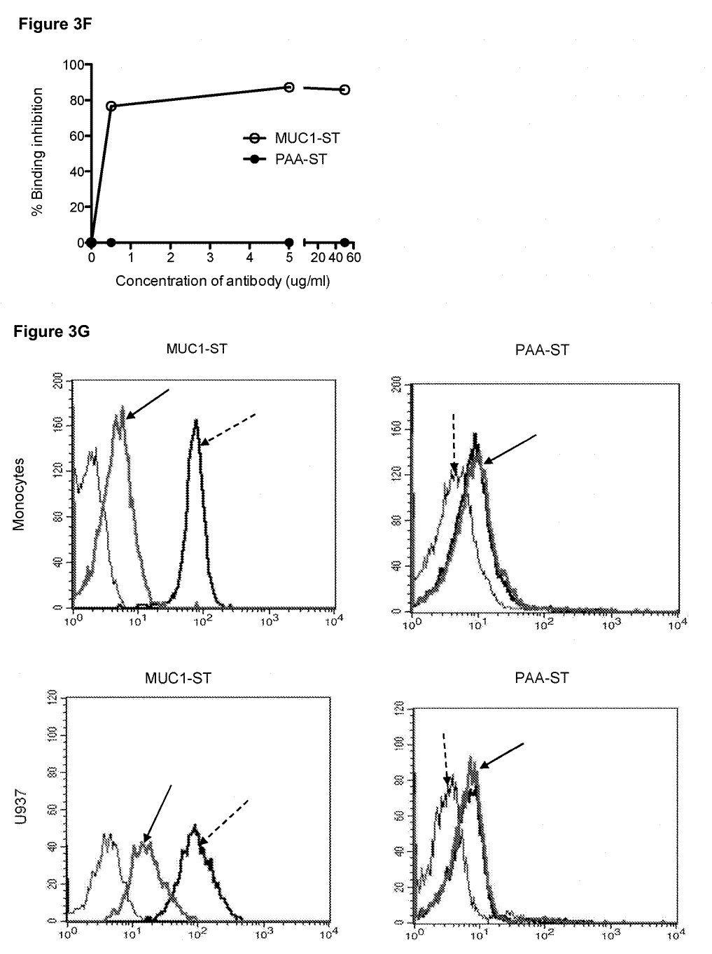

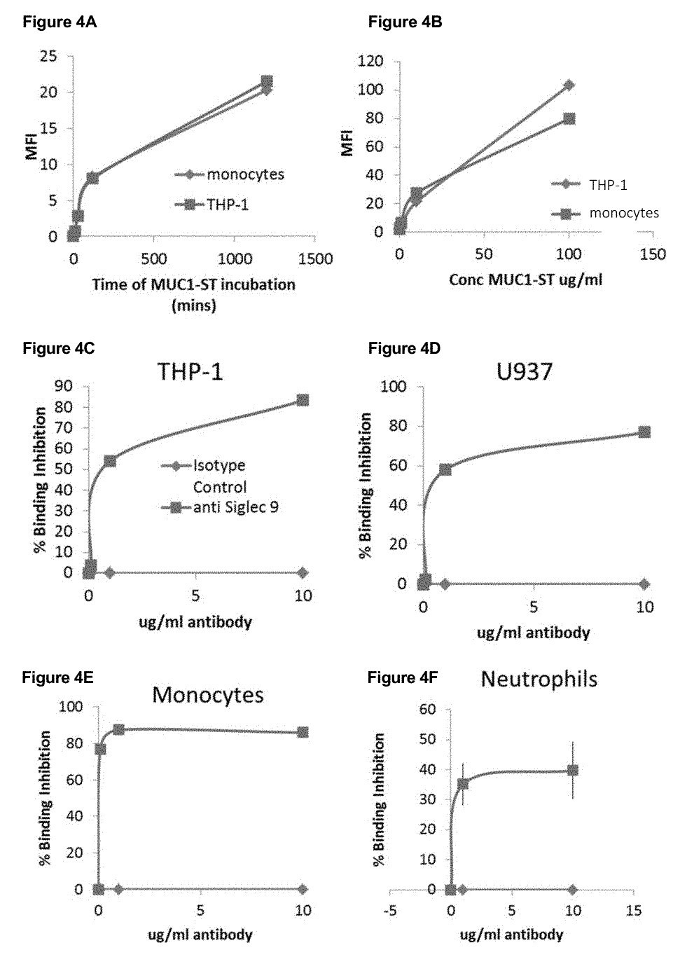

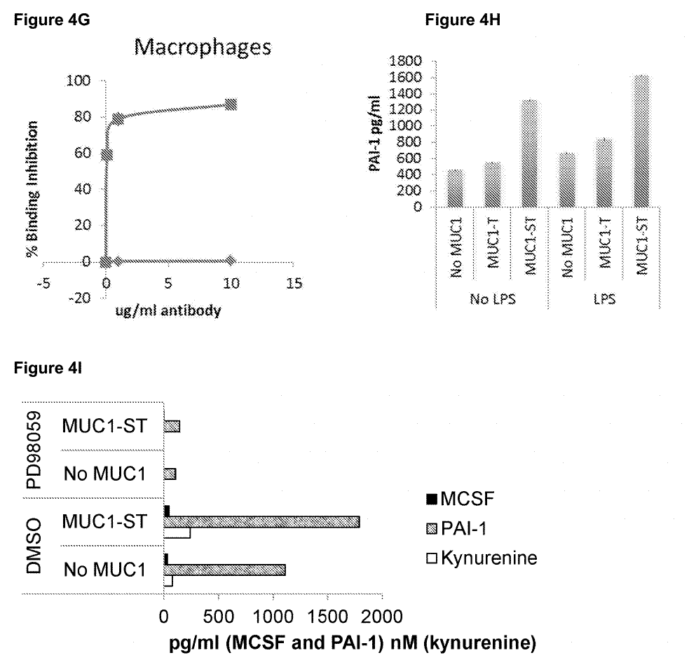

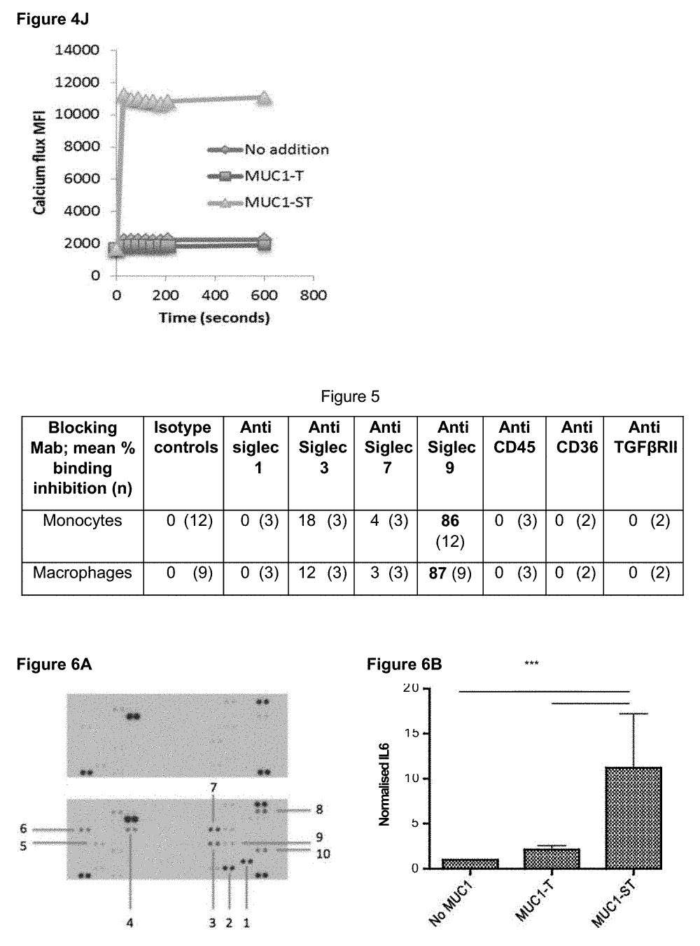

The results of interaction studies including MUC1-ST and cells of the immune system are set forth in FIG. 2. MUC1-ST was found to bind to primary monocytes and monocyte-derived macrophages and AML lines (FIGS. 2A-2B). This interaction was lost upon neuraminidase treatment of MUC1-ST demonstrating that the binding was sialic acid dependent (FIGS. 2C-2D). The binding was also time and concentration dependent (FIGS. 3A-3B) but was calcium independent (FIG. 2E). MUC1-ST was also found to bind to an established human monocytic cell line, THP-1, in both a time and concentration dependent manner (FIGS. 4A-4B).

Binding was enhanced when cells were pre-treated with 0.04 U/ml neuraminidase for 30 minutes at 37° C. in PBS (FIG. 3C), which removes competing cis-binding sialic acid sites from the surface of the cells. As this pattern is characteristic of binding to Siglecs (Macauley et al. (2014) NAT. REV. IMMUNOL. 14: 653-666), MUC1-ST binding to Siglecs was tested as follows: mouse anti human IgG was bound to plastic O/N and the plate was blocked using 1% BSA in PBS. Recombinant human Siglec (3, 5, 7, 8, 9 and 10) fusion proteins were added at 2 μg/mL for 2 hours. After incubation with 2 μg/ml biotinylated MUC1 glycoforms for 4 hours, O.D. was measured after the addition of streptavidin-HRP and substrate. It was found that MUC1-ST bound recombinant Siglecs 3, 7, 9 and 10, with the greatest binding seen for Siglec-9 (FIG. 2F). Although Siglecs 3, 7 and 9 are expressed by monocytes and macrophages (FIG. 3D), a blocking antibody to Siglec-9 inhibited 80-95% of the MUC1-ST binding to these cells (FIGS. 2G-2I, 3E, and 5) indicating this is the dominant binding Siglec. A blocking antibody to Siglec-9 also inhibited MUC1-ST binding to THP-1 and U937 cell lines (FIGS. 4C-4G). Importantly, Siglec-9 bound to the breast cancer cell line T47D that expresses MUC1 carrying sialylated Core-1 glycans (FIG. 2J). Finally, isolated monocytes were bound to 10 μg/mL biotinylated MUC1-ST or 10 μg/mL biotinylated polyacrylamide carrying the ST glycan (PAA-ST; Glycotech). The results showed polyacrylamide carrying ST glycans bound only weakly to monocytes and this could not be inhibited with anti-Siglec-9 antibody (FIGS. 3F-G). This suggests a contribution of the protein backbone to the binding specificity of Siglec-9, possibly by defining a specific spacing of the sialic acids.

### Example 2—Siglec-9 Engagement by MUC1-ST Induced the Release of Tumor-Promoting and Microenvironment Modulating Factors

The example demonstrates that the release of tumor-promoting and microenvironment modulating factors can occur following Siglec-9 engagement by MUC1-ST.

Recombinant MUC1-ST was bound to monocytes and the factors released determined using a protein array as follows. Briefly, isolated monocytes were treated with 100 μg/10⁶ cells MUC1-ST for 4 hours at 4° C., washed and incubated at 37° C. for 48 hours in AIM-V serum-free media. Supernatant was taken and cytokine production was assessed using a 102 protein array (Bio-Techne).

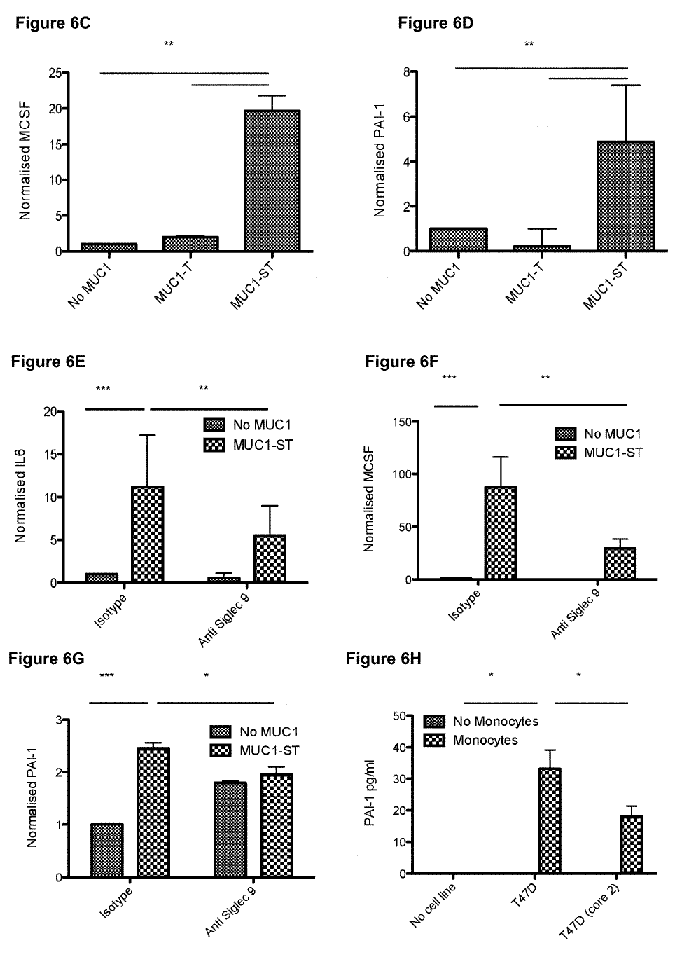

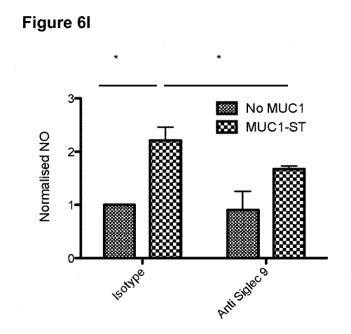

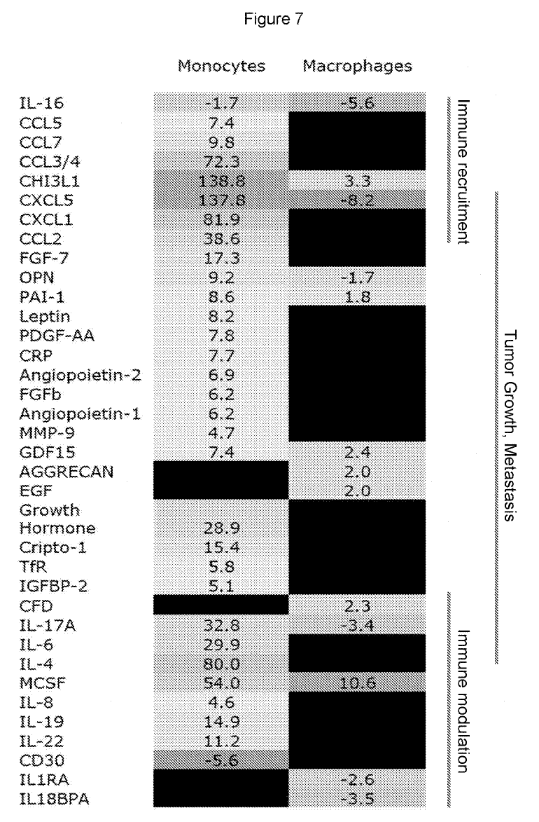

MUC1-ST induced monocytes to secrete several factors associated with inflammation and tumor progression (FIGS. 6A and 7). The induced secretion of three of these factors (IL-6, M-CSF and PAI-1 (plasminogen activator inhibitor-1)) was validated by ELISA and the induction was shown to be sialic acid (FIGS. 6B-D) and Siglec-9 dependent (FIGS. 6E-G). These factors have the potential to remodel the microenvironment by recruiting immune cells, especially monocytes and neutrophils (CXCL5, CCL2, CCL3, CXCL1, IL-8 and PAI-1) to induce angiogenesis (PAI-1, IL-8) and degrade the extracellular matrix (MMP9, PAI-1) (Jablonska et al. (2014) INT. J. CANCER 134: 1346-1358; Qian et al. (2011) NATURE 475: 222-225; Thapa et al. (2014) BIOCHEM. BIOPHYS. RES. COMMUN. 450: 1696-1701; McMahon et aL (2001) J. BIOL. CHEM. 276: 33964-33968; Bauerle et a/. (2014) J. CLIN. ENDOCRINOL. METAB. 99: E1436-E1444; Beliveau et a/. (2010) GENES DEV. 24: 2800-2811).

When monocytes were incubated with the breast cancer cell line T47D that expresses MUC1 carrying sialylated Core-1 glycan this also induced the release of PAI-1 (FIG. 6H). The secretion of PAI-1 was significantly reduced when the cells were transfected with the glycosyltransferase C2GnT1, which competes with the sialyltransferase ST3Gal-I that forms the ST glycan, resulting in ‘healthy’ branched Core-2-based side-chains that can be elongated (Dalziel et a/. (2001) J. BIOL. CHEM. 276: 11007-11015).

Similar results were seen for the human monocytic cell line, THP-1. THP-1 cells were cultured at a concentration of 1×10⁶/mL in AIM V medium. Cells were differentiated using 10 mM phorbol 12-myristate 13-acetate (PMA) on day 0 and 100 ng/mL LPS on day 3 in the presence or absence of 100 μg/mL MUC1-ST or MUC1-T. Cell supernatants were harvested on day 5 and PAI-I concentration measured by ELISA. As seen in FIG. 4H, MUC1-ST increased PAI-I secretion in differentiated THP-1 cells.

MUC1-ST was further evaluated for its ability to produce pro-inflammatory nitric oxide, a product of the arginine processing enzyme (Thompson et al. (2015) CARCINOGENESIS 36: S232-S253). Supernatant was assessed using the Griess method according to the manufacturer's instructions (Biotium). As seen in FIG. 6I, in response to MUC1-ST, monocytes produced nitric oxide.

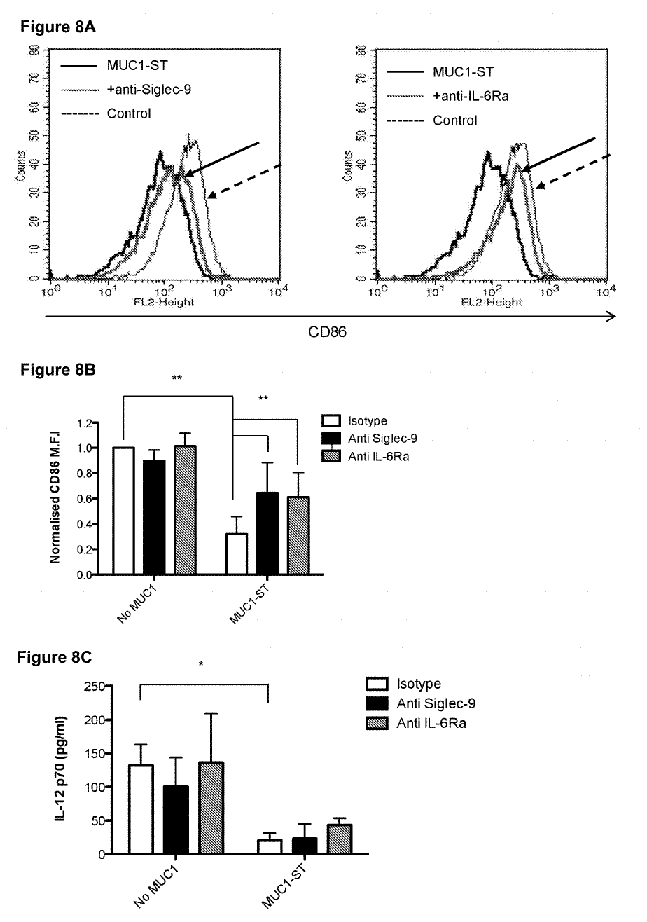

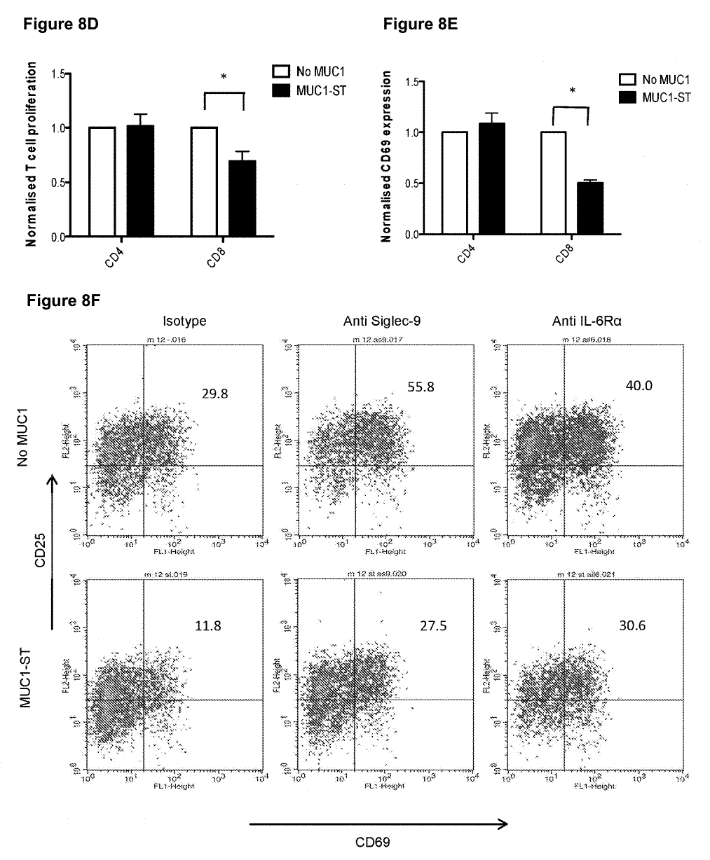

Given that IL-6 and NO are known differentiation modulators (Oosterhoff et al. (2012) ONCOIMMUNOLOGY 1: 649-658; Bogdan (2015) TREND IMMUNOL. 36: 161-178) the effects of MUC1-ST on the differentiation of monocytes into macrophages was assessed. Briefly, monocytes were differentiated into macrophages with M-CSF for seven days followed by LPS and IFNγ to give M(LPS+IFNγ) (historically defined as M1-like macrophages, see Murray et al (2014) IMMUNITY 41,14-12 for nomenclature). When MUC1-ST was added at day 1 of the culture, the differentiated macrophages displayed lower levels of the co-stimulatory molecule CD86 and IL-12 and these significant phenotypic changes could at least be partially rescued by blocking antibodies to Siglec-9 or the IL-6 receptor (FIGS. 8B-C).

In addition, primary monocytes were induced to differentiate to macrophages in the presence of MUC1-ST and then co-cultured for 48 hours with CD3/CD28 stimulated autologous CD8+ or CD4+ T cells. T cell proliferation and CD69/CD25 cell surface expression were measured using flow cytometry. The proliferation of CD8+ T cells was significantly inhibited by MUC1-ST educated M-CSF macrophages (FIG. 8D). Additionally, these CD8+ T cells showed a lower level of activation as demonstrated by the reduction of expression of CD25 and CD69 (FIGS. 8E-F). This inhibition of activation could be reversed by the presence of anti-Siglec-9 or anti-IL-6 receptor antibodies (FIG. 8F).

In addition, the effects of MUC1-ST on the differentiation of monocytes into dendritic cells were assessed. Briefly, monocytes were treated with MUC1-ST on day 0 and differentiated into immature dendritic cells (DCs) using IL-4 (1500 U/mL) and GM-CSF (400 U/mL) in AIM-V media for 6 days. Immature DCs were matured using 1 μg/mL LPS for 24 hours. Monocytes differentiated into immature DCs in the presence of MUC1-ST displayed lower levels of CD86 and, when matured, expressed lower levels of CD86 and CD83, as has been previously observed (Rughetti et al. (2005) J. IMMUNOL. 174: 7764-7772).

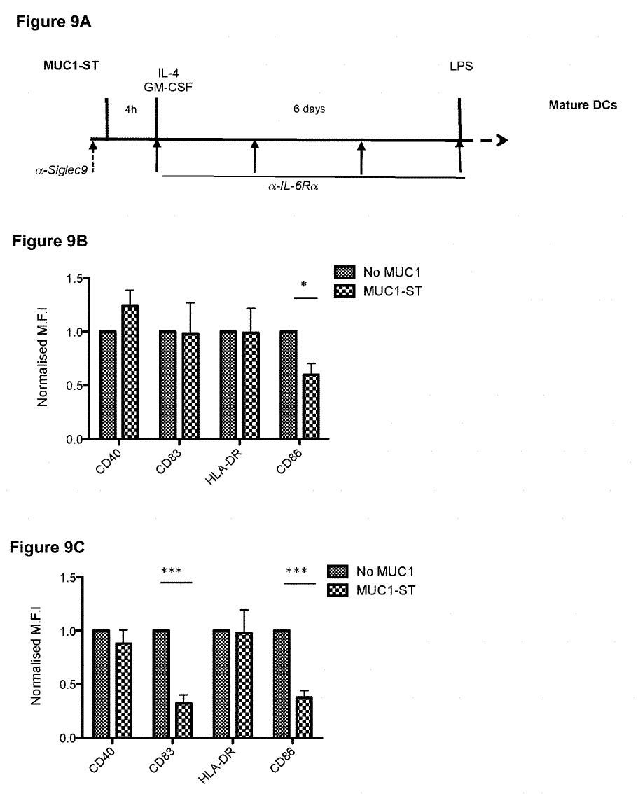

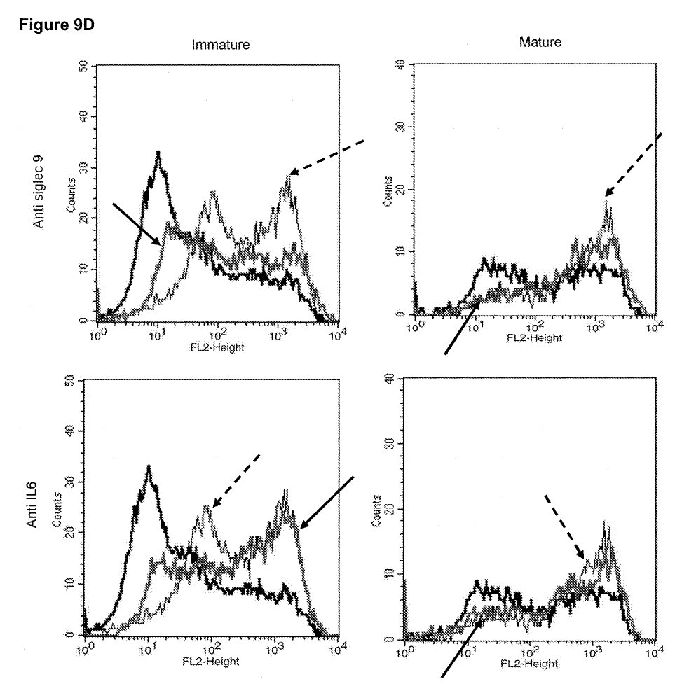

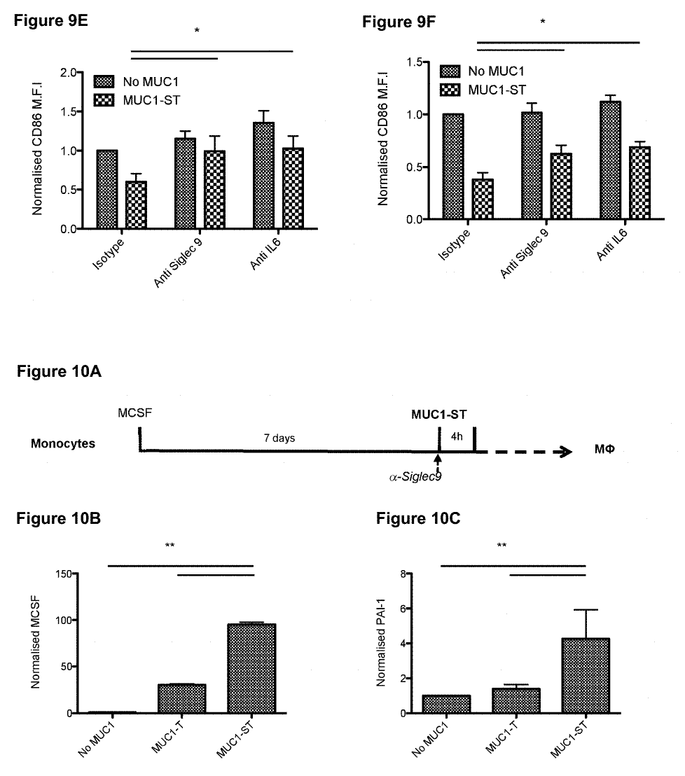

In addition, anti-Siglec-9 and IL-6 antibodies were tested to see if this effect could be reversed. Briefly, monocytes were treated with 10 μg/10⁶ cells anti-Siglec-9 antibody or isotype control before MUC1-ST treatment, prior to IL-4 and GM-CSF stimulation, or 10 μg/ml anti-IL-6Rα every 2 days as they differentiated. It was discovered that the antibodies to Siglec-9 and IL-6 could significantly reverse the effect of MUC1-ST on differentiation of dendritic cells (FIG. 9).

In summary, these results together show that MUC1-ST binding to monocytes induces a pro-inflammatory phenotype that can recruit immune cells into the site of the tumor, induce the secretion of factors associated with tumor progression and induce the differentiation of monocytes into macrophages and dendritic cells with reduced CD8 stimulatory capacity.

### Example 3—MUC1-ST Binding to Macrophages Induces a TAM-Like Phenotype

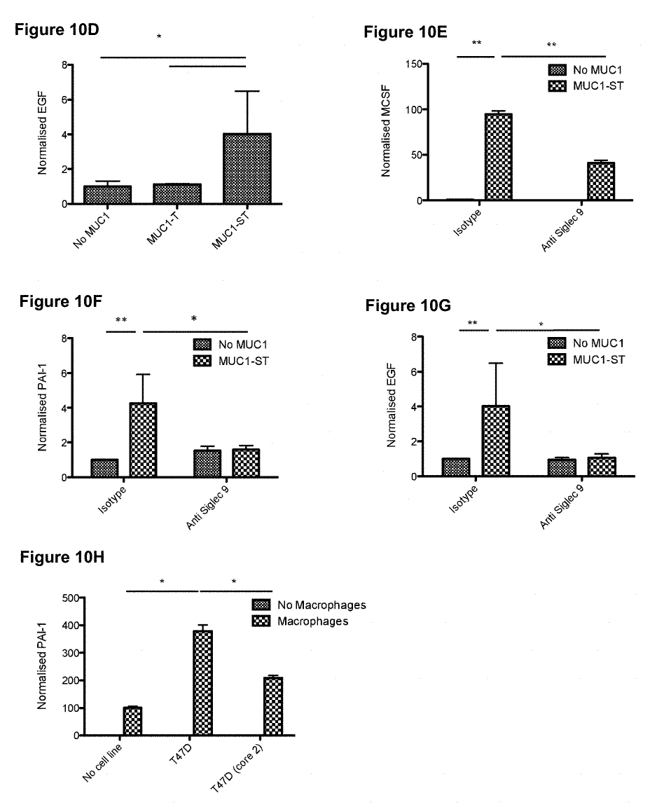

This example demonstrates that MUC1-ST binding to macrophages induces a tumor associated macrophage (TAM)-like phenotype, as shown by increased expression of CD206, CD163, IDO and PD-LI. Secreted proteins from monocyte derived macrophages were assayed by ELISA as described in Example 2. When monocyte derived macrophages were treated with MUC1-ST (as with monocytes) increased secretion of M-CSF (FIG. 10B), PAI-1 (FIG. 10C), chitinase 3-like-1 (FIG. 7), and EGF was observed (FIG. 10D). All of these factors are associated with tumor progression (Duffy et al. (2014) BREAST CANCER RES. 16: 428; Jensen et al. (2002) CLIN. CANCER RES. 9:4423-4434). Production of these factors was shown to be Siglec-9 dependent (FIGS. 10E-G). Importantly, as with monocytes, increased secretion of PM-1 after co-culturing macrophages with MUC1-ST expressing T47D cells could also be detected. Moreover, as depicted in FIG. 10H, the secretion of PAI-1 was significantly reduced when the same T47D cells were engineered to carry branched Core-2 glycans associated with normal glycosylation (Dalziel et al. (2001) J. BIOL. CHEM. 276: 11007-11015). However, unlike MUC1-ST treated monocytes, chemokines and cytokines involved in the recruitment of immune cells were decreased or did not change (FIG. 7). It has been discovered that MUC1-ST/Siglec-9 ‘educated’ monocytes and macrophages have a unique secretome.

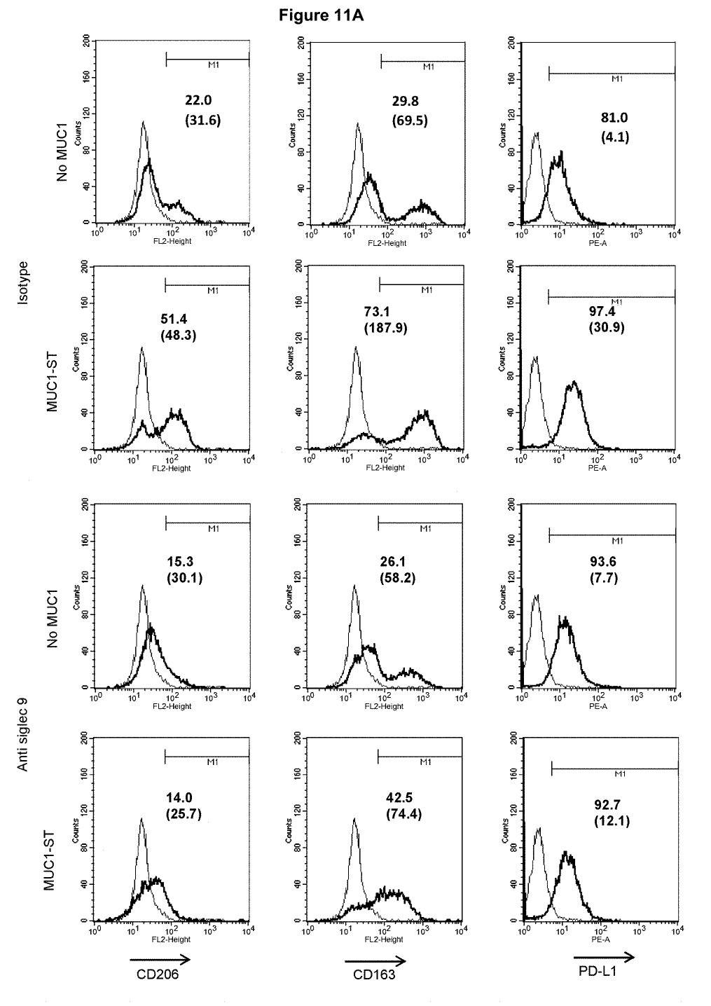

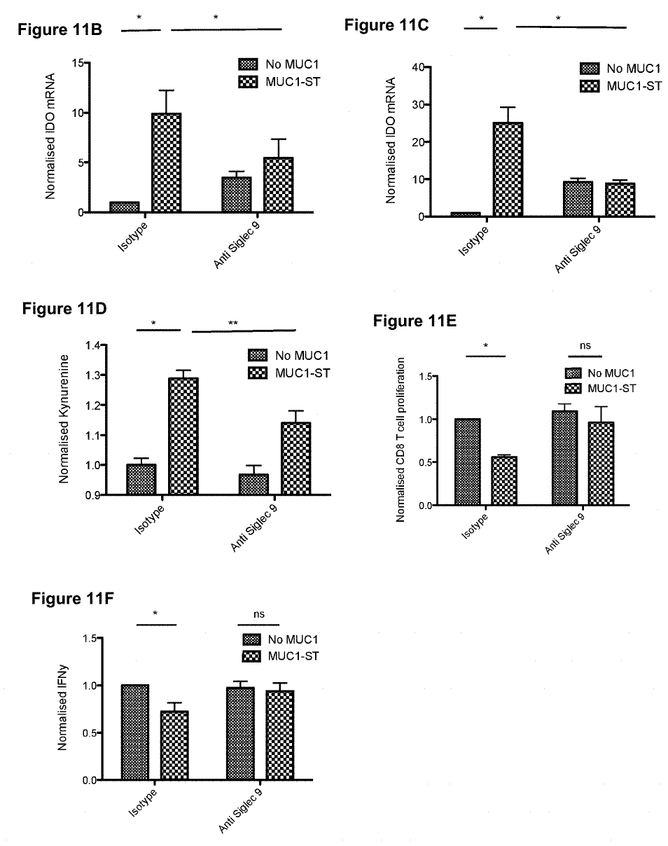

When the phenotype of MUC1-ST treated macrophages was investigated, these cells showed increased levels of mannose receptor (CD206) and the scavenger receptor CD163 (FIG. 11A), which are tumor-associated macrophage markers. Moreover, increased expression of the immune checkpoint ligand PD-L1 was observed (FIG. 11A). These phenotypic changes could all be rescued by competing out the binding of MUC1-ST to macrophages with an antibody to Siglec-9 (FIG. 11A).

In addition, treatment of macrophages with MUC1-ST increased the expression of the mRNA encoding indoleamine 2,3-dioxygenase (IDO) by 10-25 fold (FIGS. 11B-C), which again could be rescued using a Siglec-9 antibody. Given that IDO catalyzes the rate-limiting step in the metabolism of tryptophan, the tryptophan metabolite kynurenine was detected as follows. 604, supernatant was mixed with 304, 30% trichloroacetic acid (TCA) and incubated for 30 minutes at 50° C. The supernatant was spun at 3000×g and 50 μL was harvested and mixed with 50 μL freshly prepared Ehrlich Reagent (2% p-dimethylaminobenzaldehyde in glacial acetic acid). After 10 minutes optical density (O.D.) was measured at 492 nm, and concentrations were calculated against a kynurenine standard curve. An increase in the tryptophan metabolite kynurenine was observed (FIG. 11D).

IDO activity inhibits proliferation and induces apoptosis of T cells (Forouzandeh et al. (2008) MOL. CELL BIOCHEM. 309: 1-7). Moreover increased expression of PD-L1 can engage the PD-1 receptor on activated T cells inhibiting their function (Gianchecchi et al. (2013) AUTOIMMUN. REV. 12: 1091-100). Indeed, the data showing that MUC1-ST binding to Siglec-9 can increase expression of PD-L1 by macrophages is an important observation as immune checkpoint inhibitors are showing extremely promising results in the clinic (Garon et al. (2015) N. ENGL. J. MED. 372: 2018-28). The degree of increase in expression of PD-LI does differ with donors and ranges from 1.5 fold to over 7 fold. Highly relevant to this is that even modest effects on the expression of PD-L1 can lead to dramatic results (Casey et al. (2015) SCIENCE 352: 227-231) so changes up to 7 fold have the potential to be highly relevant to tumor growth.

Thereafter, the effects of MUC1-ST educated macrophages on T cell function were analyzed. Macrophages treated with MUC1-ST were co-cultured with eFluor® 670 labelled allogeneic CD8+ T cells in the presence of absence of anti-Siglec-9 antibody or isotype control. Indeed, macrophages that had been educated with MUC1-ST were decreased in their ability to stimulate the proliferation of allogeneic CD8 T cells (FIG. 11E). Moreover, decreased CD8 IFNγ secretion was observed, which could be inhibited with anti-Siglec-9 blocking antibody. (FIG. 11F). This profile of expression and functional activity is indicative of tumor-associated macrophages (TAMs), which play a role in promoting tumor progression (Noy et al. (2014) IMMUNITY 41: 49-61; Sousa et al. (2015) BREAST CANCER RES. 17: 101; Qian et al. (2010) CELL 141: 39-45).

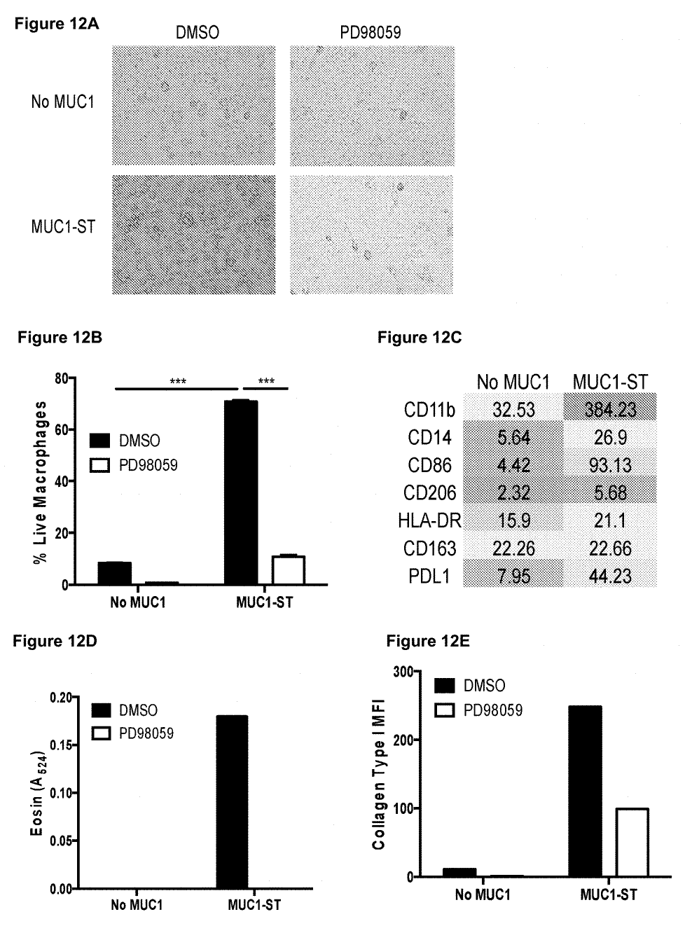

To further explore the role of MUC1-ST in inducing a TAM-like phenotype, monocytes from PBMCs were plated in serum-free medium, incubated with MUC1-ST or PBS, and cultured for 7 days. Imaging and visual analysis of live macrophages as well as eosin staining revealed that MUC1-ST increased the percentage of live macrophages in the culture (FIG. 12A-12B). Phenotyping of the cells using flow cytometry indicated an increased expression of TAM markers such as CD206 and PD-L1 in the presence of MUC1-ST (FIG. 12C). TAMs are also associated with extracellular matrix (ECM) deposition, and MUC1-ST induced increased expression of the ECM component collagen type I (FIG. 12E). These results indicate that MUC1-ST alone can induce a TAM phenotype in monocytes.

Together, these results identify MUC1-ST as a novel myeloid modulating factor and as a new driver of TAM formation demonstrated by the increased expression of CD206, CD163, IDO and PD-L1. Additionally, these macrophages with a TAM-like phenotype can inhibit the proliferation and activation of CD8+ T cells. Moreover, engagement of Siglec-9 on monocytes and macrophages by this tumor-associated glycoform of MUC1 induces the increased secretion of proteins involved in disease progression. Thus this MUC1-ST/Siglec-9 axis plays an important role in orchestrating a tumor-permissive environment.

Given that tumor derived MUC1-ST can enhance the expression of the PD-L1 and IDO in macrophages in MUC1-ST/Siglec-9 mediated manner, it is contemplated that enhanced anti-tumor activity may be potentiated using an agent that prevents the binding of MUC1-ST to Siglec-9 (for example, an anti-Siglec-9 neutralizing antibody) in combination with an immune checkpoint inhibitor (for example, an anti-PD-L1 neutralizing antibody or an anti-PD-1 neutralizing antibody) and/or an IDO inhibitor.

### Example 4—MUC1-ST Binding to Siglec-9 Induces Calcium Flux Leading to MEK/ERK Activation

This example demonstrates that MUC1-ST binding to Siglec-9 induces calcium flux can lead to MEK/ERK activation.

To determine the intracellular effects of MUC1-ST binding to Siglec-9, the ability of MUC1-ST to induce phosphorylation of the immunoreceptor tyrosine-based inhibitory motif (ITIM) of Siglec-9 thereby inducing intracellular inhibitory signals (Avril et al. (2004) J. IMMUNOL. 173: 6841-6849) was assessed. Without wishing to be bound by theory, it was hypothesized that this was likely occur as the repeated glycans found on MUC1 could be able to crosslink this lectin. To investigate the effects of MUC1-ST on Siglec-9 phosphorylation, monocytes or differentiated M-CSF macrophages were treated with MUC1-ST or cross-linked anti-Siglec-9 antibody at 4° C. for 4 hours or 30 minutes, respectively, and were then brought to 37° C. for 15 minutes, and lysed in the presence of pervanadate. Lysates were assessed for the phosphorylation of Siglec-9 using an ELISA or a 59 phospho immunoreceptor array (Bio-Techne) according to the manufacturer's instructions. For the ELISA, anti-human Siglec-9 was plated overnight (O/N) on plastic before being blocked with 1% BSA in PBS. Clarified supernatant was added and incubated for 2 hours. After incubation with 1 μg/mL biotinylated anti phospho-tyrosine, O.D. at 450 nm was measured after the addition of streptavidin-HRP and substrate.

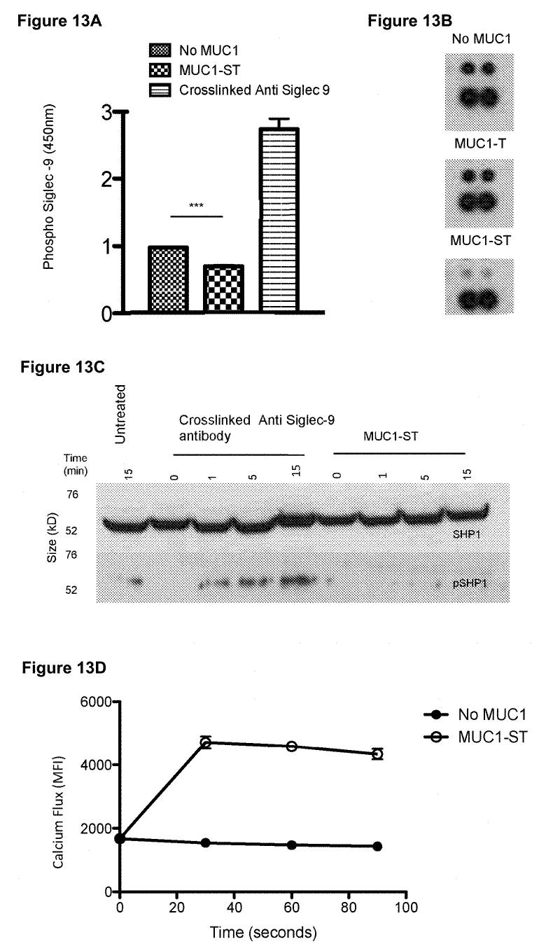

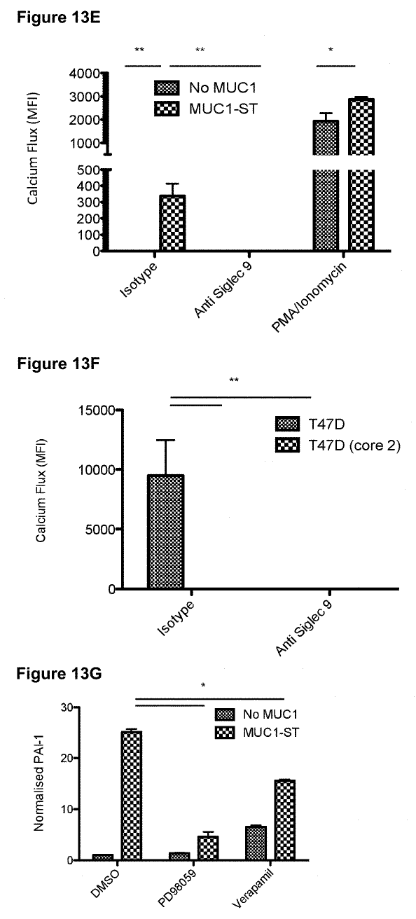

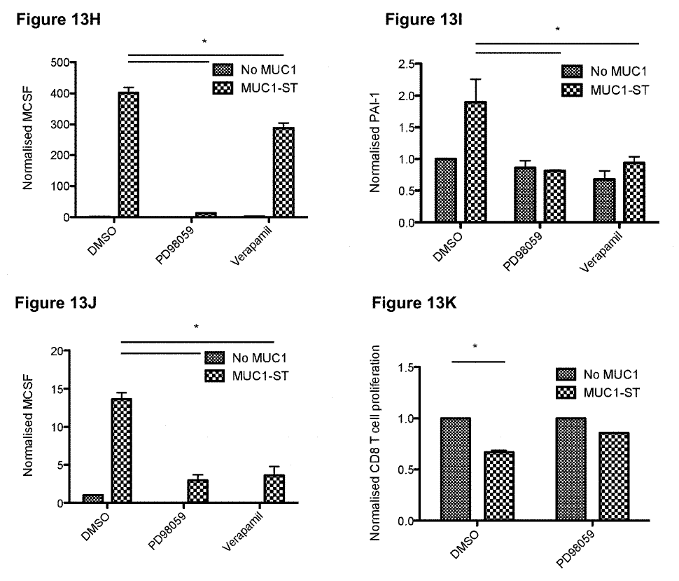

It was discovered that, instead of promoting phosphorylation, MUC1-ST inhibited the resting phosphorylation of Siglec-9 in monocytes and macrophages (FIGS. 13A-B). Importantly crosslinking of an anti-Siglec-9 antibody induced phosphorylation (FIG. 13A).

A Western blot to assay phosphorylation of SHP, which is recruited by phosphorylated Siglec-9 (Avril et al. (2004) supra) was conducted. Monocytes were incubated with MUC1-ST and lysed as described above, and the resulting lysates were separated by SDS PAGE (10% gel) before being transferred, blocked and probed with anti-SHP1 (Santa Cruz), anti-phospho SHP1 (Abcam) and appropriate secondary antibodies. Phosphorylation of SHP was not observed after MUC1-ST binding to Siglec-9 on primary monocytes (FIG. 13C) although again, phosphorylation of SHP1 was observed when Siglec-9 was activated via antibody cross-linking. No activation of SHP2 was observed. This is in contrast to other unknown ligands on tumor cells, whose engagement with Siglec-9 has been shown to result in SHP1 recruitment.

In addition, in a murine tumor model Siglec-E (the mouse Siglec with the most similarity to human Siglec-9), was associated with a decrease in alternatively activated macrophages (Laubli et al. (2014) PROC. NATL. ACAD. SCI. USA). As a result, the triggering of a calcium flux when MUC1-ST engaged Siglec-9 was investigated. Briefly, monocytes pre-labeled with an intracellular calcium reporter (Fluo-4; Life Technologies) were treated with MUC1-ST, MUC1-T (100 μg/10⁶ cells) or a T47D monolayer, for 4 hours at 4° C. The cells were brought up to 37° C. and calcium flux was measured at 530 nm using a plate reader at the indicated time points. Where not indicated, the time point was 60 seconds. When monocytes or macrophages were treated with MUC1-ST, a Siglec-9 dependent increase in calcium influx was observed (FIGS. 13E-F). A calcium flux was also observed when monocytes and T47D cells came into contact. This effect could also be inhibited by the anti-Siglec-9 antibody. Furthermore, as seen in FIG. 13F, the increase in calcium flux was not seen when the same cells were engineered to carry normal branched Core-2 glycans (Dalziel et al. (2001) J BIOL. CHEM. 276: 11007-11015).

As binding of MUC1-ST to Siglec-9 did not induce phosphorylation associated with inhibitory signalling but rather induced a calcium flux, which is associated with activating signals (Xuan et al. (2014) PATHOL. ONCOL. RES. 20: 619-624), the downstream signalling pathway following MUC1-ST binding to Siglec-9 was investigated. To explore this, the secretion of PAI-1 and M-CSF from MUC1-ST educated monocytes and macrophages was measured following treatments with 1 μM PD98059 or 20 μM verapamil for 20 minutes at 37° C., where indicated. The secretion of PAI-1 and M-CSF was found to be significantly inhibited by calcium channel inhibitor verapamil (FIG. 13G-J).

Intracellular calcium flux can lead to activation of the MEK/ERK pathway (Christo et al. (2015) IMMUNOL. AND CELL BIOLOGY 93: 694-704). When monocytes or macrophages were incubated with MUC1-ST in the presence of the highly selective MEK inhibitor PD9805943 secretion of PAI-1 and M-CSF was significantly inhibited (FIGS. 13G-J). Moreover, the repression of T cell proliferation by MUC1-ST treated macrophages could be overcome when MEK signalling was inhibited in the macrophages treated with MUC1-ST (FIG. 13K). Furthermore, treatment with the MEK inhibitor PD98059 at 10 μM inhibited MUC1-ST mediated TAM formation in monocytes (FIGS. 12B, 12D-12E).

The intracellular effects of MUC1-ST binding to Siglec-9 were further explored in the monocytic cell line, THP-1. THP-1 cells were cultured for three days at a concentration of 1×10⁶/mL in AIM V medium and differentiated using 10 mM phorbol 12-myristate 13-acetate (PMA) in the presence or absence of 100 μg/mL MUC1-ST. Calcium flux was measured as described above, and MUC1-ST was found to induce calcium flux in THP-1 cells (FIG. 4J). THP-1 cells were further treated with DMSO or the MEK/ERK inhibitor PD98059 at 1004, and the concentration of PAI-1, M-CSF and kynurenine in cell supernatants were measured as described above. Consistent with earlier results, MUC1-ST increased PAI-1, kynurenine, and, to a lesser extent, M-CSF concentration in THP-1 cell supernatants. This increase in concentration was blocked by the MEK/ERK inhibitor PD98059 (FIG. 4I).

Together, these results demonstrate a novel activating role for Siglec-9. In contrast to classical Siglec engagement, which results in the recruitment and activation of the phosphatases SHP-1 or SHP-2, Siglec-9 engagement by MUC1-ST does not induce phosphorylation of this Siglec or SHP1, but induces the transmission of activating signals. The mechanism whereby MUC1-ST binding to Siglec-9 on monocytes and macrophages acts as an immune modulator inducing changes in the tumor microenvironment to promote tumor growth is via the induction of a calcium flux leading to activation of the MEK/ERK pathway.

### Example 5—Diagnostic Applications of Siglec-9 Activity

To further investigate the link between Siglec-9 activity and cancer, formalin fixed paraffin embedded primary breast cancer samples will be stained for PD-L1, IDO, and CD206 on macrophages, which will then be correlated with MUC1-ST expression in the breast cancer cells. MUC1-ST expression will be assayed by staining for MUC1-T with and without neuraminidase treatment as described herein above. Digitalized slides will be used for image analysis with HistoQuest 4.2, where the HistoQuest algorithms use haematoxylin and eosin (H&E) staining to differentiate cell populations based on cell size and nuclear shape. Correlation based on intensity and spatial antigen expression will be assessed through automated random selection of regions of interest for quantification. It is contemplated that expression of MUC1-ST by the epithelial cancer cells correlates with expression of TAM markers on macrophages infiltrating into the tumor.

A correlation of PAI-1 and CHI3L1 present in sera from the breast cancer patients with tumors expressing MUC1-ST can also be analyzed. It is contemplated that MUC1-ST expression by the cancer cells would correlate with PAI-1 and CHI3L1 secreted into serum as both these factors are induced to be secreted by monocytes and macrophages after exposure to MUC1-ST. PAI-1 and CHI3L1 have both previously been correlated with a poor prognosis in cancer patients.

In addition, cancers, such as breast cancer may be disaggregated using either enzymes or the GentleMacs dissociator and the phenotype of the tumor-associated macrophages determined by flow cytometry and correlated with the expression of MUC1-ST by the cancer cells.

### Example 7—Therapeutic Applications of Siglec-9 Inhibitors

To further investigate the use of inhibitors of Siglec-9 activity in cancer therapy, neutralizing antibodies to Siglec-9, with or without MER/ERK or calcium flux inhibitors, will be tested to determine if the neutralizing antibodies can inhibit the migration of immune cells induced by MUC1-ST educated macrophages. Monocytes will be induced to secrete chemokines by co-culture with the breast cancer cell line T47D that expresses MUC1 carrying sialylated Core-1 glycans (MUC1-ST) or control T47D cells engineered to carry branched Core-2 glycans. The migration of added labeled monocytes or neutrophils in the presence or absence of anti-Siglec-9 antibodies, MERK/ERK inhibitors, or calcium flux inhibitors is then measured. It is contemplated that anti-Siglec-9 antibodies and calcium channel/MEK/ERK inhibitors inhibit the migration of monocytes and neutrophils towards the MUC1-ST educated monocytes.

An organotypic breast cancer model derived from tissue slices may also be used, in particular to investigate the effects of Siglec-9 blockade on the induction of a TAM-like phenotype. This model preserves the morphology and structure of the original tumor. Media from the breast cancer slices can be cultured for 5 days in the presence or absence of an inhibitor of Siglec-9 activity, and will then be assayed for M-CSF, PAI-1 and CH3L1. It is contemplated that the presence of the inhibitor reduces TAM markers. In addition, FFPE sections made from the cultured slices may be stained to assess macrophage phenotype and MUC1-ST expression on the tumor cells.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-63. (canceled)

64. An inhibitor of Siglec-9 activity for use in the treatment of a cancer, wherein the cancer comprises cancerous cells that express one or more sialylated Core-1-MUC1 glycoproteins.

65. The inhibitor of claim 64, wherein the one or more sialylated Core-1-MUC1 glycoproteins comprise MUC1-ST, MUC1-diST, or a combination thereof.

66. The inhibitor of claim 64, wherein the inhibitor acts by blocking, reducing or otherwise neutralizing binding between the one or more sialylated Core-1-MUC1 glycoproteins and Siglec-9.

67. The inhibitor of claim 64, wherein the inhibitor is an antibody, an aptamer, a spiegelmer, an anti-sense molecule, a small molecule, or a combination thereof; and/or the inhibitor is a small molecule that is a MEK/ERK inhibitor or a calcium flux inhibitor.

68. The inhibitor of claim 64, wherein the inhibitor is an anti-Siglec-9 antibody; and/or the inhibitor is an anti-Siglec-9 antibody having a binding affinity greater than 1 nM for Siglec-9; and/or the inhibitor is an anti-Siglec-9 antibody having a human IgG1, IgG2, IgG3, IgG4, or IgE isotype.

69. A combination comprising the inhibitor of claim 64 and an IDO inhibitor or an immune checkpoint inhibitor for use in the treatment of cancer; the immune checkpoint inhibitor being a PD-1 inhibitor, PD-L1 inhibitor, CTLA-4 inhibitor, adenosine A_{2A} receptor inhibitor, B7-H3 inhibitor, B7-H4 inhibitor, BTLA inhibitor, KIR inhibitor, LAG3 inhibitor, TIM-3 inhibitor, VISTA inhibitor, or TIGIT inhibitor.

70. A method of treating cancer in a subject in need thereof, the method comprising administering to the subject an effective amount of an inhibitor of Siglec-9 activity thereby to treat the cancer in the subject, wherein the cancer has been identified as comprising cancerous cells that express one or more sialylated Core-1-MUC1 glycoproteins.

71. The method of claim 70, wherein the subject is a human subject.

72. The method of claim 70, wherein the one or more sialylated Core-1-MUC1 glycoproteins comprise MUC1-ST, MUC1-diST, or a combination thereof; and Siglec-9 is expressed by a monocyte or a macrophage in the subject.

73. The method of claim 70, further comprising administering an IDO inhibitor or an immune checkpoint inhibitor, the immune checkpoint inhibitor being a PD-1 inhibitor, PD-L1 inhibitor, CTLA-4 inhibitor, adenosine A_{2A} receptor inhibitor, B7-H3 inhibitor, B7-H4 inhibitor, BTLA inhibitor, KIR inhibitor, LAG3 inhibitor, TIM-3 inhibitor, VISTA inhibitor, or TIGIT inhibitor.

74. A method of reducing PDL-1 or IDO expression in (i) a monocyte or macrophage or (ii) a neutrophil that expresses Siglec-9 and is capable of binding a sialylated Core-1-MUC1 glycoprotein expressed by a cancerous cell, the method comprising contacting (i) the monocyte or macrophage or (ii) the neutrophil with an inhibitor of Siglec-9 activity thereby to reduce PDL-1 or IDO expression in (i) the monocyte or macrophage or (ii) neutrophil.

75. The method of claim 74, wherein the sialylated Core-1-MUC1 glycoprotein comprises MUC1-ST, MUC1-diST, or a combination thereof; and/or the sialylated Core-1-MUC1 glycoprotein is secreted from the cancerous cell and/or expressed on the cell surface of the cancerous cell.

76. The method of claim 74, wherein the cancerous cell is derived from or associated with breast, colon, colorectal, lung, ovarian, pancreatic, prostate, cervical, endometrial, head and neck, liver, renal, skin, stomach, testicular, thyroid, or urothelial cancer; and/or the cancerous cell is an adenocarcinoma, derived from or associated with a metastatic cancer; and/or the cancerous cell is derived from or associated with a refractory cancer.

77. The method of claim 74, wherein the inhibitor prevents differentiation of a macrophage into a tumor-associated macrophage (TAM); and/or the inhibitor induces the macrophage to differentiate into a pro-inflammatory macrophage or prevents the loss of pro-inflammatory activity; and/or the inhibitor reduces upregulation of indoleamine 2,3-dioxygenase (IDO), CD163, CD206, or PD-L1 expression in the macrophage or the TAM; and/or the inhibitor acts by blocking, reducing or otherwise neutralizing binding between the sialylated Core-1-MUC1 glycoprotein and Siglec-9.

78. The method of claim 74, wherein the inhibitor is an antibody, an aptamer, a spiegelmer, an anti-sense molecule, or a small molecule, or a combination thereof, the small molecule being a MEK/ERK inhibitor or a calcium flux inhibitor.

79. The method of claim 74, wherein the inhibitor is an anti-Siglec-9 antibody; and/or the inhibitor is an anti-Siglec-9 antibody having a binding affinity greater than 1 nM for Siglec-9; and/or the inhibitor is an anti-Siglec-9 antibody having a human IgG1, IgG2, IgG3, IgG4, or IgE isotype.

80. A method of identifying a subject with cancer likely to respond to treatment with an inhibitor of Siglec-9 activity, wherein the method comprises determining whether cancer cells obtained from the subject express one or more sialylated Core-1-MUC1 glycoproteins, the one or more sialylated Core-1-MUC1 glycoproteins comprising MUC1-ST, MUC1-diST, or a combination thereof.

81. The method of claim 80, wherein the cancerous cells are present in a tissue or body fluid sample harvested from the subject; and/or the subject is a human subject.

82. The method of claim 80, wherein the one or more sialylated Core-1-MUC1 glycoproteins are expressed on the cell surface of the cancerous cells and/or are secreted from the cancerous cells; and the cancer is breast, colon, lung, ovarian, pancreatic or prostate cancer, an adenocarcinoma, metastatic cancer, refractory cancer, or a combination thereof.

83. The method of claim 80, where the presence of the one or more sialylated Core-1-MUC1 glycoproteins is determined using an antibody; the antibody being specific for Core-1-MUC1 glycoproteins;

84. The method of claim 83, wherein binding of the antibody before and after treatment with a neuraminidase enzyme is determined and/or quantified, and a difference in binding is attributed to the presence of the one or more sialylated Core-1-MUC1 glycoproteins.


SEQUENCE LISTING (4 sequences)

SEQ ID NO: 1 - protein, 20 aa, Homo sapiens
GSTAPPAHGVTSAPDTRPAP

SEQ ID NO: 2 - protein, 1233 aa, Homo sapiens
GSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 3 - protein, 127 aa, Homo sapiens
KLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPN

SEQ ID NO: 4 - protein, 445 aa, Homo sapiens
TSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR